SEQUENCE LISTING (15 sequences)

SEQ ID NO: 1 - DNA, 196 bp, Artificial Sequence
  RNAi sequence R2D2egfp
ggctagctagctctagaggatccgtggttgctgatgagtccgtgaggacgaaacggtacccggtaccgtccaaccactacctgagcacccagttcaagagactgggtgctcaggtagtggttgtcgacggatcatgatccgtcctgatgagtccgtgaggacgaaacaaccacgaattcaagcttgacctctcgac

SEQ ID NO: 2 - DNA, 196 bp, Artificial Sequence
  RNAi sequence R2D2xera
ggctagctagctctagaggatcccttgccgctgatgagtccgtgaggacgaaacggtacccggtaccgtccggcaagctgaccctgaagttcttcaagagagaacttcagggtcagcttgccgtagacggatcatgatccgtcctgatgagtccgtgaggacgaaacggcaaggaattcaagcttgacctctcgac

SEQ ID NO: 3 - DNA, 190 bp, Artificial Sequence
  RNAi sequence R2D2Non
ggctagctagctctagaggatcccggagaactgatgagtccgtgaggacgaaacggtacccggtaccgtcttctccgaacgtgtcacgtttcaagagaacgtgacacgttcggagaattgacggatcatgatccgtcctgatgagtccgtgaggacgaaattctccggaattcaagcttgacctctcgac

SEQ ID NO: 4 - DNA, 21 bp, Artificial Sequence
  PCR amplification primer R2D2For
ggctagctagctctagaggat

SEQ ID NO: 5 - DNA, 21 bp, Artificial Sequence
  PCR amplification primer R2D2Rev
gtcgagaggtcaagcttgaat

SEQ ID NO: 6 - DNA, 107 bp, Artificial Sequence
  EGFP targeting construct R2D2xer1
ggctagctagctctagaggatcccttgccgctgatgagtccgtgaggacgaaacggtacccggtaccgtccggcaagctgaccctgaagttcttcaagagagaactt

SEQ ID NO: 7 - DNA, 110 bp, Artificial Sequence
  EGFP targeting construct R2D2xer2
gtcgagaggtcaagcttgaattccttgccgtttcgtcctcacggactcatcaggacggatcatgatccgtctacggcaagctgaccctgaagttctctcttgaagaactt

SEQ ID NO: 8 - DNA, 107 bp, Artificial Sequence
  EGFP targeting construct R2D2egfp1
ggctagctagctctagaggatccgtggttgctgatgagtccgtgaggacgaaacggtacccggtaccgtccaaccactacctgagcacccagttcaagagactgggt

SEQ ID NO: 9 - DNA, 110 bp, Artificial Sequence
  EGFP targeting construct R2D2egfp2
gtcgagaggtcaagcttgaattcgtggttgtttcgtcctcacggactcatcaggacggatcatgatccgtcgacaaccactacctgagcacccagtctcttgaactgggt

SEQ ID NO: 10 - DNA, 104 bp, Artificial Sequence
  Non-targeting construct R2D2non1
ggctagctagctctagaggatcccggagaactgatgagtccgtgaggacgaaacggtacccggtaccgtcttctccgaacgtgtcacgtttcaagagaacgtga

SEQ ID NO: 11 - DNA, 107 bp, Artificial Sequence
  Non-targeting construct R2D2non2
gtcgagaggtcaagcttgaattccggagaatttcgtcctcacggactcatcaggacggatcatgatccgtcaattctccgaacgtgtcacgttctcttgaaacgtga

SEQ ID NO: 12 - DNA, 423 bp, Artificial Sequence
  Sequence from the pCDNA3.1 vector containing 5′ and 3′ cis-acting ribozymes with RNAi targeting EGFP
aaattttcnnnccccccaaaaaaanacctttttccctagctctaaaggacccggagaactgatgagtccgtgaggacgaaacggtacccggtaccgtcttctccgaacgtgtcacgtttcaagagaacgtgacacgttcggagaattgacggatcatgatccgtcctgatgagtccgtgaggacgaaattctccggaattcaagcttaagtttaaaccgctgatcagcctcgactgtgccttctagttgccagccntctnttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggnaagaca

SEQ ID NO: 13 - DNA, 446 bp, Artificial Sequence
  Sequence from the pcDNA3.1 vector carrying 5′ and 3′ cis-acting ribozymes and the non-targeting RNAi control sequence
cnngnttcaggcccnnacnnaatttttcnnncnccccaananaaaanacccttttttccctagctctagaaggatccgtgggttgctgatgaagtccgtgaaggacgaaacggtacccggtaccgtccaaccactacctgagcacccagttcaagagactgggtgctcaggtagtggttgtcgacggatcatgatccgtcctgatgagtccgtgaggacgaaacaaccacgaattcaagcttaagtttaaaccgctgatcagcctcgactgtgccttctagttgnnngncatctgttgtttgcccctcccccgtgccttccttgaccctggaaggtgccactcccactgtcctttcctaataaaatgaggaaattgcatcgcattgtctgaagtaggtgtcattctattctggggggtggggtggggcaaggacagcaagggggga

SEQ ID NO: 14 - DNA, 395 bp, Artificial Sequence
  Sequence from pcDNA3.1 with the H1 promoter driving expression of RNAi targeting EGFP
tccnattaccctccctaaagggacaaaagctnggagctccaccgcggtggcggccgctctagaactagtggatcccccgggctgcaggaattcgaacgctgacgttcatcaacccgctccaaggaatcgcgggcccagtgtcactaggcgggaacacccagtgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatccccaaccactacctgagcacccagntcaagagactgggtgctcaagtagtggtttttttggaaaacttatcgataccgtct

SEQ ID NO: 15 - DNA, 352 bp, Artificial Sequence
  Sequence from the pcDNA3.1 vector carrying sequence for the rat albumin promoter
ctggcttcgaattatcnnctcctatagggagnccaagctggctcgcgtttaacgggccctctagactcgagcggccgctctagcttccttagcatgacgttccacttttttctaaggtggagcttacttctttgatttgatcttttgtgaaacttttggaaattacccatcttcctaagcttctgcttctctcagttttctgcttgctcattccattttccagctgacctgccccctaccaacattgctccacaagcacaaattcatccnnagaaaataaattctaagttttatagttgtttggatcgcataggtagctaaagaggtggcaacccacacatccttaggcatg

(19) United States
(12) Patent Application Publication
(10) Pub. No.: US 2004/0198967 A1
(43) Pub. Date: Oct. 7, 2004

(54) Compositions and methods for tissue specific or inducible inhibition of gene expression

(75) Inventors: Danny Allen, Dublin (IE); Gwenyth Jane Farrar, Monkstown (IE)

Correspondence Address: TESTA, HURWITZ & THIBEAULT, LLP, HIGH STREET TOWER, 125 HIGH STREET, BOSTON, MA, 02110, US

(73) Assignee: Provost, Fellows and Scholars of The College of The Holy and Undivided Trinity of Queen Elizabeth, Dublin (IE)

(21) Appl. No.: 10/655,570

(22) Filed: Sep. 4, 2003

Related U.S. Application Data
(60) Provisional application No. 60/408,210, filed on Sep. 4, 2002.

(51) Int. Cl. A61K 48/00; C07H 21/04

(52) U.S. Cl. 536/23.1; 514/44

(57) ABSTRACT

Methods and reagents are provided for the tissue specific, cell specific and/or inducible expression of RNAi. The invention may be used to downregulate the expression of endogenous or exogenous RNAs targeted by the RNAi in cells, animals and/or plants.

### RELATED APPLICATIONS

This application claims priority to U.S. Provisional Application Serial No. 60/408,210, filed Sep. 4, 2002, the entire disclosure of which is incorporated herein by reference.

### FIELD OF THE INVENTION

The invention relates generally to compositions and methods for the tissue specific and/or cell specific and/or inducible expression of RNAis.

### BACKGROUND OF THE INVENTION

A new tool for modulating or suppressing gene expression has been described called interfering RNA (RNAi) or small interfering RNA (siRNA) or double stranded RNA (dsRNA) (Fire, A. et al. (1998) Nature 391: 806-811; Jorgensen, R. A. et al. (1996) Plant Mol. Biol. 31(5): 957-973; Caplen, N. J. et al. (2001) Proc. Natl. Acad. Sci. USA 98: 9742-9747; Elbashir, S. M. et al. (2001) Nature 411: 494-498; Yang, S. et al. (2001) Mol. Cellul. Biol. 21: 7807-7816; Paddison, P. J. et al. (2002) Proc. Natl. Acad. Sci. 99: 1443-1448; Krichevsky, A. M. et al. (2002) 99: 11926-11929; Lewis, D. L. et al. (2002) Nature Genetics 32: 107-108; Zamore, P. D. (2001) Nature Structur. Biol. 8: 746-750; Clemens, M. J. et al. (1997) J. Interferon Cytokine Res. 17: 503-524; Miyagishhi, M. et al. (2002) Nature Biotechnol. 20: 497-500; Paul, C. P. et al. (2002) Nature Biotech. 20: 505-508; Sui, G. et al. (2002) Proc. Natl. Acad. Sci. 99: 5515-20; Jacque, J. M. et al. (2002) Nature 418: 435-438). Identified in C. elegans by Andrew Fire and colleagues (Fire, 1998), the applications for this biological tool have now been extended to many species and RNAi has been shown to be effective in both mammalian cells and animals (Elbashir (2001) Nature 411:494). An important feature of RNAi is that it is double stranded RNA that lacks large overhanging pieces of single stranded RNA, although RNAi with small overhangs or intervening loops of RNA has been used to suppress a target gene. RNAi administered in vitro and in vivo as pre-synthesized RNA or expressed from a viral or non-viral vector is functional (Lewis (2002) Nature Genetics 32:107; Miyagishhi (2002) Nature Biotechnology 20:497; Paul (2002) Nature Biotech 20:505; Siu (2002) RNAS 99:5515). Additionally, RNAi has been used to generate transgenic animals expressing RNAi (McCaffrey et al. (2003) Nature Biotech 21:639, McManus et al. (2003) Nature Genet. 33:401, Sharp et al. (2003) RNA 9:493.

The pathway for silencing gene expression involving long (>30 nucleotides) double stranded RNA molecules has been elucidated and is thought to work via the following steps (shown in Drosophila melanogaster) (Zamore, 2001). Firstly, long RNAi is cleaved into RNAi approximately 21 nucleotides in length. This RNAi targets complimentary mRNA sequence, which is degraded. However, in mammals long RNAi triggers a non-specific response causing a decrease in all mRNA levels. This general suppression of protein synthesis is mediated by a RNAi dependent protein kinase (PKR) (Clemens, 1997). Elbashir et al. were able to specifically suppress target mRNA with 21 nucleotide RNAi duplexes. Notably, RNAi bypassed the non-specific pathway and allowed for gene-specific inhibition of expression (Elbashir, 2001; Caplen, 2001).

Promoters, such as, for example, the U6 or H1 promoters have been used to express RNAi in multiple cell(s) types (Miyagishhi, 2002; Paul, 2002; Sui, 2002). Typically, pol III promoters are used to express RNAi in vitro and in vivo. However, the utility of RNAi as a suppression tool has been limited because such promoters do not provide tissue or cell specific expression of RNAi. One reason for the lack of tissue or cell specificity is that the transcription start site (TSS) of many tissue specific, cell specific and inducible promoters is not clearly defined. In addition, transcription by a tissue specific, cell specific or inducible promoter may result in, or require, the transcription of sequences 3′ to the TSS (i.e., 5′ to the desired RNAi sequence)—the inclusion of which sequences in the transcript can inhibit RNAi folding and/or activity. For example, for translated RNAs, these sequences would be the 5′ and 3′ untranslated regions (UTRs) that may be required for tissue specific, cell specific or inducible expression of the RNA. In essence transcription from some promoters can result in sequences between the TSS and the beginning of the expressed sequence being transcribed. Moreover, termination signals for eukaryotic genes can vary significantly from relatively simple termination signals used by some polIII promoters, which typically involve a run of uridines, to complex signals, such as multiple poly adenylation (polyA) signals.

A need therefore exist for compositions and methods for the tissue specific, cell specific, and/or inducible expression of RNAi.

### SUMMARY OF THE INVENTION

The present inventors have overcome a number of problems associated with the prior art and have enabled, for the first time, the tissue specific, cell specific, and inducible expression of RNAi.

The present invention thus enables the suppression of gene expression by RNAi that is tailored to specific tissues. Suppression of a target gene in all or many tissues of an organism may be lethal. Furthermore, suppression of a target gene in all or many tissues of an organism may prevent the elucidation of the biological function of a given gene in a particular tissue or cell type.

The invention thus enables RNAi-based therapies with optimal safety profiles by enabling the limitation of expression of therapeutic RNAis to specific tissues. Further, the invention further allows not only tissue specific control of expression of the suppression agent, RNAi, but also control of the level and timing of expression of RNAi using, for example, inducible promoters.

Moreover the invention further enables methods that direct the controlled synthesis of specific RNAi sequences in a tissue specific, cell specific and inducible manner that does not necessarily require prior knowledge of the transcription start site(s) of a gene.

Accordingly, in a first aspect of the invention, there is provided a polynucleotide comprising a nucleotide sequence encoding an RNAi operatively linked to a tissue specific promoter, a cell specific promoter, and/or an inducible promoter.

In an embodiment, the promoter is expressed exclusively in a single cell type. Alternatively, a tissue or cell specific promoter that drives transcription in more than one but less than all cell types (e.g., the promoter drives transcription only in a subset of cell types) may be used. In a preferred embodiment, the promoter is a tissue or cell specific promoter.

In an embodiment, the promoter is an inducible promoter that is capable of driving the expression of an RNAi in response to an applied stimulus. Such stimuli may include, but are not limited to, inflammatory mediators, growth factors, hormones, drugs, heat, and light, for example.

In a preferred embodiment, the promoter is a RNA polymerase II promoter. For example, particularly preferred promoters for use in the invention include collagen lAl promoters, collagen 1A2 promoters, collagen 3A1 promoters, cone transducin alpha subunit GNAT-2 promoters, peripherin-retinal degeneration slow (rds) promoters, rhodopsin promoters, cone arrestin promoters, RPE65 promoters, Thyrotropin releasing hormone (TRH) promoters, THR-degrading ecotoenzymes promoters, TRH receptor promoters, albumin promoters, insulin promoters, Huntington's promoters, presenillin 1 and 2 promoters, superoxide dismutase (SOD) promoters, and enolase promoters (Table 2).

In preferred embodiments, the polynucleotide further comprises a cleaving element that is capable of cleaving a nucleotide sequence 5′ and/or 3′ to the RNAi sequence. Any suitable cleaving element may be used in the invention, for example a ribozyme, a maxizyme, or a minizyme DNAzyme. Preferably, the cleaving element is a ribozyme. The cleaving element is preferably located 3′ of the promoter sequence driving expression. The cleaving element can itself be located 5′ and/or 3′ of the termination signal(s) for transcription.

In another embodiment the polynucleotide further comprises one or more cleaving elements that are capable of cleaving a nucleotide sequence 5′ and/or 3′ and/or between the sense and the antisense strands of the RNAi sequence.

In one embodiment, the sense and antisense strands of RNAi are expressed each using a promoter and sequences encoding sense and antisense arms of RNAi are present on the same and/or different constructs. Promoters to express sense and antisense strands of RNAi can be the same and/or different promoters. Antisense and sense expression cassettes can have 5′ and/or 3′ cleaving element(s).

The cleaving element is preferably operatively linked to a tissue specific promoter and/or a tissue specific and/or an inducible promoter. Although in some embodiments of the invention the promoter to which the cleaving element is operatively linked is different than that to which the RNAi is operatively linked, in preferred embodiments of the invention the cleaving element is operatively linked to the same promoter as the RNAi. The cleaving element may be cis-acting or trans-acting. In a preferred embodiment the cleaving element is a cis-acting ribozyme. In another embodiment, the invention utilizes one or more 5′ and/or 3′ cis-acting ribozymes and/or other cleaving elements to release RNAi.

In preferred embodiments, the polynucleotide may further comprise at least one suppression agent (e.g., a trans-acting ribozyme) capable of suppressing one or more target gene(s) or nucleotide sequences.

The polynucleotide may further comprise a transcription termination sequence. In a particular embodiment, a transcription termination sequence utilized by Poll and/or PolIII promoters (for example, a run of uridines) is utilized together with a 5′ and/or 3′ cis-acting ribozyme. In another embodiment, at least one transcription termination sequence similar to those utilized for polII promoters, e.g., sequences involving poly adenylation signals, is used in conjunction with one or more 5′ and/or 3′ cis-acting ribozymes. In another embodiment, a combination of PolI and/or PolII and/or PolIII termination signals is utilized in conjunction with cis-acting ribozymes.

In a preferred embodiment, one or more 5′ cleaving elements is used in conjunction with a minimal poly A termination signal at the 3′ of the transcript.

The inclusion of biological tools to cleave transcripts 5′ and 3′ and/or between the sequence encoding sense and antisense strands of the expressed RNAi provides flexibility in sequence requirements 5′ and 3′ of the RNAi thereby enabling greater flexibility in choice of sequences controlling initiation and termination of transcription.

In a preferred embodiment of the invention, the RNAi sequence is placed at or close to the transcription start site(s) (TSS(s)) of the promoter. In this embodiment of the invention, RNAi is expressed from a tissue and/or cell specific and/or inducible promoter such that the expressed sequence is close to, or directly adjacent to, one or more TSSs of the promoter. In an embodiment, the RNAi sequence is within 1 to 1000, within 1 to 50, preferably within 1 to 25, more preferably within 1 to 15, more preferably within 1 to 10, most preferably within 1 to 5 bases of one or more TSSs of the promoter. The RNAi sequence to be expressed may incorporate the TSS.

In an embodiment of the invention, sequences 5′ and/or 3′ of the TSSs of the promoters described are modified such that transcription of sequences expressed from these promoters can be initiated from TSSs that lie close to or juxtaposed to the sequence to be expressed.

In another embodiment of the invention, the tissue or cell specific or inducible promoters described can use more than one TSS when transcribing a sequence in vitro and/or in vivo. The particular TSS utilized at a given time can be influenced, for example, by cellular cues and/or sequence context.

In another embodiment of the invention, the tissue or cell specific or inducible promoter(s) described use single TSS for all, or the majority, of transcription events driven from a particular promoter.

In another embodiment of the invention, the TSSs used by tissue or cell specific or inducible promoter(s) described need not be fully characterised.

In preferred embodiments of the invention, the RNAi sequence comprises a first region complementary or partially complementary to a target gene, a second region complementary or partially complementary to the first region and a spacer region separating the first and second regions. The spacer region can be 1-10 bases, 10-100 bases or 100-1,000 bases in length. In particularly preferred embodiments of the invention, the RNAi sequence is capable of discriminating between different alleles of the same gene.

According to a second aspect of the present invention, there is provided a vector comprising a polynucleotide according to the first aspect of the invention.

The vectors of the invention may be viral, non-viral, an artificial chromosome or any vehicle for delivery of the RNAi nucleotides. In an embodiment, the RNAi sequence(s) and the cleaving element sequence(s) (if present) are on the same vector. In an alternative embodiment, the RNAi sequence(s) and the cleaving element sequence(s) are on different vectors. The RNAi encoding sequence(s) and cleaving element sequence(s) may be expressed from different promoters or from the same promoter, e.g., as a single RNA.

According to a third aspect of the present invention, there is provided a host cells comprising the polynucleotide of the first aspect of the invention or the vector according to the second aspect of the invention. The polynucleotide is preferably integrated into the host cell genome.

According to a fourth aspect of the invention, transgenic organisms are provided comprising the polynucleotide according to the first aspect of the invention, the vector according to the second aspect of the invention and/or the host cell according to the third aspect of the invention. The transgenic organism is preferably non-human, more preferably a non-human mammal.

In a fifth aspect of the invention, there is provided a method of inhibiting or reducing expression of a target gene in a cell of an organism, the method comprising the steps of: (i) administering to the cell a polynucleotide according to the first aspect of the invention or the vector according to the second aspect of the invention, wherein the RNAi has specificity for the target gene; and (ii) allowing expression of the RNAi such that the RNAi inhibits or reduces expression of the target gene.

Preferably, the polynucleotide is integrated into the genome of the cell.

The present invention may also be used in assays to identify and test putative modulators of gene expression. Thus, in a sixth aspect of the invention, there is provided a method of identifying a modulator of a target gene, the method comprising the steps of:

(i) providing a host cell of the invention or a transgenic organism of the invention;

(ii) administering a candidate modulator to said host cell or said transgenic organism; and

(iii) determining expression of said target gene in the presence of the candidate modulator.

Also provided is a pharmaceutical composition comprising a polynucleotide of the invention, and/or a vector of the invention and/or a host cell of the invention, and/or a pharmaceutical excipient.

Further provided by the invention is a polynucleotide according to the first aspect of the invention, and/or a vector according to the second aspect of the invention and/or a host cell according to the third aspect of the invention for use in a method of medical treatment or diagnosis.

The invention further provides use of a polynucleotide according to the first aspect of the invention, a vector according to the second aspect of the invention and/or a host cell according to the third aspect of the invention in the preparation of a medicament for the treatment of retinitis piginentosa, epidermolysis bullosa, osteogenesis imperfecta, Ehlers-Danlos syndrome, Marfan's disease, dominant negative cancers, Alzheimer's disease, motor neuron disease, poly cystic kidney disease, or a disorder due to poly glutamine expansions such as Huntington's chorea.

The invention further provides use of a polynucleotide according to the first aspect of the invention, a vector according to the second aspect of the invention, or a host cell according to the third aspect of the invention in the preparation of a medicament for the treatment and/or modulation of a disease pathology such that one or more of the feature(s) associated with the pathology are modulated, for example, apoptosis, which is associated with many disorders, for example, neurological disorders. Similarly the invention may be used to modulate components orchestrating wound healing, a feature associated with many disorders.

### BRIEF DESCRIPTION OF THE DRAWINGS

The foregoing and other objects, features and advantages of the present invention, as well as the invention itself, will be more fully understood from the following non-limiting description of preferred embodiments when read together with the accompanying drawings, in which:

FIG. 1 shows exemplary RNAi constructs carrying one or more cis-acting ribozymes. One or more cis-acting ribozymes can be included in RNAi expressing vectors and can be used to flank 5′ and/or 3′ either one or both strands of the stretch of nucleotides that encodes the RNAi. The inclusion of sequence for one or more cis-acting ribozyme(s) 5′ and/or 3′ to the RNAi to be expressed enables flexibility in choice of sequences used to control initiation and termination of transcription. Sense and antisense stretches of nucleotides that contribute to RNAi may be generated such that they are physically unlinked or are linked by a loop of nucleotides not less than 1 and not more than 10,000 nucleotides in length.

FIG. 2 shows exemplary RNAi constructs carrying a RNAi sequence to be expressed adjacent one or more TSSs of a tissue specific and/or cell specific and/or inducible promoter. Also shown is a construct carrying a promoter fused to a RNAi sequence close or at one or more TSSs of the promoter together with a cis-acting ribozyme 3′ of the RNAi sequence to be expressed.

FIG. 3 provides a list of eukaryotic promoters that have been shown experimentally to drive tissue specific or cell specific expression of a transgene either in cell culture and/or in vivo.

FIG. 4A shows the design of an RNAi targeting EGFP and a non-targeting RNAi control. FIG. 4B shows the design of RNAi targeting EGFP and a non-targeting RNAi control with cis-acting hammerhead ribozymes 5′ and 3′ of the RNAi sequence. Arrows highlight ribozyme cleavage sites. FIG. 4C shows the design of the 5′/3′ ribozyme-EGFP RNAi cassette when driven by a CMV promoter and cloned into pcDNA3.1 (Invitrogen-Cat.# V79520). FIG. 4D shows sequence from the pcDNA3.1 vector containing 5′ and 3′ cis-acting ribozymes with RNAi targeting EGFP. FIG. 4E shows sequence from the pcDNA3.1 vector carrying 5′ and 3′ cis-acting ribozymes and the non-targeting RNAi control sequence. Table 1 provides sequences for oligonucleotides that were utilized to generate ribozyme-RNAi constructs. FIG. 4F shows sequence from pcDNA3.1 with the HI promoter driving expression of RNAi targeting EGFP. FIG. 4G shows sequence from the pcDNA3.1 vector carrying sequence for the rat albumin promoter.

FIG. 5A provides the design of a ribozyme-RNAi cassettes targeting EGFP driven by a liver specific promoter. FIG. 5B provides the design of a ribozyme-RNAi cassettes targeting EGFP driven by a liver specific promoter cloned into a vector also expressing the EGFP target (from a CMV promoter). FIG. 5C provides the design of a ribozyme-RNAi cassettes targeting EGFP driven by a photoreceptor specific promoter (the GNAT-2 promoter).

FIG. 6 shows designs for RNAi constructs with 5′ and/or 3′ ribozymes and/or ribozymes between the sense and antisense strands of the sequence encoding RNAi. Sequences encoding sense and antisense strands of RNAi can be driven by the same and/or separate promoters and/or can be found on the same or separate constructs.

FIG. 7 shows the design of a tissue specific RNAi cassette where RNAi sequences are placed directly beside or adjacent to the TSS of the tissue specific and/or cell specific and/or inducible promoter. For example, 3.8 kb of the mouse rhodopsin promoter up to the TSS is utilized to drive photoreceptor specific expression of RNAi placed directly adjacent (from 0-10 bases) and 3′ to the rhodopsin TSS.

### DETAILED DESCRIPTION OF THE INVENTION

RNAi

Throughout this application, the terms RNAi, dsRNA and RNAi are used interchangeably.

Polynucleotides of the invention comprise a nucleotide sequence encoding an RNAi, a short double stranded RNA molecule that comprises a double stranded region that is identical or nearly identical in sequence to a target gene nucleic acid sequence that the RNAi is capable of silencing or inhibiting. The RNAi may be blunt ended or may have overhangs at its 3′ or 5′ termini. The overhangs are preferably short in length, for example less than 30 nucleotides, preferably less than 20 nucleotides, more preferably less than 10 nucleotides, even more preferably less than 5 nucleotides, most preferably less than 3 nucleotides in length. In a preferred embodiment, the overhangs are two nucleotides in length.

Typically, the region of the RNAi sequence with sequence identity to the target gene is from 14 to 30 nucleotides in length, for example from 16 to 24 nucleotides, more preferably from 18 to 22 nucleotides, most preferably from 19 to 21 nucleotides in length.

Promoters

The expression of the RNAi in the polynucleotides of the invention is driven by tissue specific, cell specific and/or inducible promoters. Any suitable promoters may be used. The choice will depend on the selectivity and specificity of tissue expression required.

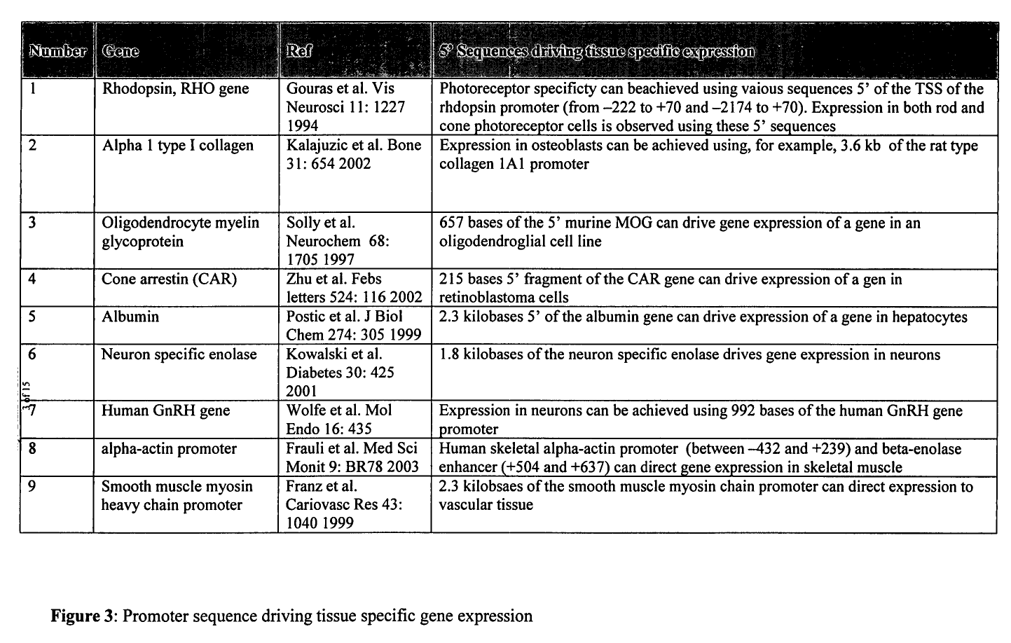

Many sequences 5′ and 3′ of a gene or nucleotide sequence have been described that can be used to elicit tissue specific and/or cell specific and/or inducible expression of a gene or nucleotide sequence (Bennett, J. et al. (1998) Gene Ther. 5(9): 1156-64; Ying, S. et al. (1998) Curr. Eye Res. 17(8): 777-82; Tannour-Louet, M. et al. (2002) Hepatology 35(5): 1072-81; Follenzi, A. et al. (2002) Hum. Gene Ther. 13(2): 243-60; Lee, M. et al. (2001) J. Control Release 10 75(3): 421-29; Lottmann, H. et al. (2001) J. Mol. Med. 79(5-6): 321-28; Georgopoulos, S. et al. (2002) Biochem. 30 41(30): 9293-301; Phillips, M. I. et al. (2002) Hypertension 39(2 Pt 2): 651-55; Reynolds, P. N. et al. (2001) Nature Biotechnol. 19(9): 838-42; Sakai, N. et al. (2002) Mol. Pharmacol. 61(6): 1453-64; Utomo, A. R. et al. (1999) Nature Biotechnol. 17(11): 1091-96). For example, tissue specific promoters enabling expression in diverse tissues such as photoreceptors, hepatocytes, pancreas, brain, heart and many other cell types have been described (Bennett, 1998; Ying, 1998; Tannour-Louet, 2002; Follenzi, 2002; Lee, 2001; Lottmann, 2001; Georgopoulos, 2002; Phillips, 2002). Various features of some promoters (such as enhancer sequences) that can give rise to a given promoter's tissue and level specificity have been defined (see FIG. 3, for example).

The majority of eukaryotic promoters are termed polII promoters, so called because they are transcribed using the polII RNA polymerase, although, pol I and polIII promoters are utilized to express some eukaryotic genes. The DNA sequence features or control elements of polI, polII and polIII promoters are well known in the art. Typical polI promoters include features such as CpG-rich regions around the transcriptional start site (TSS), a sequence motif TATAAA (TATA box) around position −30 relative to the TSS and two GC-rich regions around the TATA box. Although these features are typical, they are not required features of polI promoters. Standard methods well known to the skilled artisan, such as 5′-RACE and primer extension, can be used to determine the exact TSS of a promoter. More than one TSSs may be used by a promoter (Zhu et al. FEBS letters 524:116 2002% the particular TSS active at any point in time depends, for example, on specific cellular cues and/or on the context of the surrounding DNA sequence. The Eukaryotic Promoter Database (www.epd.isb-sib.ch) and services/programmes such as PromoSer (biowulf.bu.edu/zlab/PromoSer/), PromoterInspector and Eponine may also provide information on the TSS of a given promoter.

Promoters that may be used in the present invention include, but are not limited to collagen 1A1, collagen 1A2, GNAT-2, peripherin-rds, rhodopsin, retinal pigment epithelium 65 (REP65) promoters, cone arrestin promoters, albumin, insulin, huntington, collagen3A1, super oxide dismutase promoters, presenillin1 and 2 promoters, enolase promoters. (See Table 2)

TABLE 2A
List of Genes (abbreviations and in full) with promoters that may be
used in the invention to drive tissue specific expression of RNAi.

| Gene | Gene name in full |
|---|---|
| ABCA4 | ATP-binding cassette transporter |
| ABO | Blood group antigen |
| ADA | Adenosine deaminase deficiency |
| ADRB3 | Beta-3 adrenergic receptor |
| AIPL1 | Aryl hydrocarbon receptor-interacting |
| | protein-like 1 |
| ALB | Albumin |
| ALDH (1B1, 2, 4, 9, | Aldehyde dehydrogenase |
| 3A1, 3A2) | (1B1, 2, 4, 9, 3A1, 3A2) |
| APC | Adenomatous polyposis coli |
| AR | Androgen receptor |
| AT3 | Antithrombin |
| ATM | Ataxia-telangiectasia |
| BCP | Blue cone pigment |
| BLM | Bloom syndrome |
| BRCA1 | Breast cancer 1 |
| BRCA2 | Breast cancer 2 |
| CDKN2A | Cyclin-dependent kinase inhibitor |
| CFTR | Cystic fibrosis |
| CHS1 | Chediak-Higashi syndrome |
| CLN (1, 2, 3, 5, 8) | Ceroid lipofuscinosis |
| CNGA1 | Cyclic nucleotide-gated cation channel 1 |
| CNGA3 | Cyclic nucleotide-gated cation channel 3 |
| COL1A1, COL1A2, | Collagen (types I and III) |
| COL3A1 | |
| CRB1 | Crumbs homologue 1 |
| CRX | Cone rod homeobox |
| CYBA | Chronic granulomatous disease |
| CYBB | X-linked chronic granulomatous disease |
| DMD | Duchenne muscular dystrophy |
| EMD | Emery-Dreifuss muscular dystrophy |
| FANCA, FANCC | Fanconi anaemia |
| FBN1 | Fibrillin 1 |
| FBN1 | Fibrillin 2 |
| F7 | Factor VII |
| F8 | Factor 8 |
| F9 | Factor 9 |
| GAA | Acid alpha-glucosidase |
| GCH1 | GTP cyclohydrolase 1 deficiency |
| GCP | Green cone pigment |
| GNAT1 | |
| HEXA | Hexosaminidase A |
| HEXB | Hexosaminidase B |
| HPS | Hermansky-Pudlak |
| L1CAM | L1 cell adhesion molecule |
| MEFV | Mediterranean fever |
| MEN1 | Multiple endocrine neoplasia 1 |
| MYO7A | Myosin VIIa |
| NAT1, NAT2 | Arylamine N-acetyltransferases |
| NBS1 | Nijmegen breakage syndrome 1 |
| NCF1 | Chronic granulomatous disease 1 |
| NCF2 | Chronic granulomatous disease 2 |
| NF1 | Neurofibromatosis type 1 |
| NF2 | Neurofibromatosis type 2 |
| NR2E3 | Photoreceptor cell-specific nuclear receptor |
| NRL | Neuroretina-linked leucine zipper |
| NYX | Nyctalopin |
| OA1 | Ocular albinism 1 |
| OCA2 | Ocular albinism 2 |
| OTC | Ornithine transcarbamylase deficiency |
| PAH | Phenylketonuria |
| PCBD | Pterin-4a-carbinolamine dehydratase deficiency |
| PDC | Phosducin |
| PDE6A, PDE6B | Phosphodiesterase type 6 |
| PLP | Proteolipid protein |
| PPT1 | Palmitoyl-protein thioesterase |
| PRKCG | Protein kinase C gamma |
| PRNP | Prion Protein |
| PROC | Protein C |
| PROS | Protein S |
| PROML1 | Prominin (mouse)-like 1 |
| PSEN1, PSEN2 | Presenilin 1 and Presenilin 2 |
| PTS | 6-Pyruvoyl-tetrahydropterin synthase deficiency |
| QDPR | Dihydropteridine reductase deficiency |
| RB1 | Retinoblastoma |
| RBP4 | Retinol-binding protein 4 |
| RCP | Red cone pigment |
| RDH5 | 11-cis retinol dehydrogenase |
| RDS | Retinal degeneration, slow |
| RGR | RPE-retinal G-protein-coupled receptor |
| RHO | Rhodopsin |
| RHOK | Rhodopsin kinase |
| RLBP1 | Cellular retinaldehyde-binding protein |
| ROM1 | Rod outer membrane protein 1 |
| RP1 | Retinitis pigmentosa 1 |
| RP2 | Retinitis pigmentosa 2 |
| RPE65 | Retinal pigment epithelium specific protein |
| RPGR | Retinitis pigmentosa GTPase regulator |
| RS1 | Retinoschisis 1 |
| SGCA | Sarcoglycan-alpha |
| SGCB | Sarcoglycan-beta |
| SGCG | Sarcoglycan-gamma |
| SGCD | Sarcoglycan-delta |
| TIMP3 | Tissue inhibitor of metalloproteinase 3 |
| TSC1 | Tuberous sclerosis 1 |
| TSC2 | Tuberous sclerosis 2 |
| TTR | Transthyretin |
| TULP1 | Tubby-like protein 1 |
| TYR | Tyrosinase |
| TYRP1 | Tyrosinase-related protein 1 |
| USH2A | Usher syndrome 2A |
| VHL | Von Hippel-Lindau |
| VMD2 | Vitelliform macular dystropy |
| VWF | Von Willebrand disease |
| WRN | Werner syndrome |
| WT1 | Wilm's tumor |

TABLE 2B
List of Inducible Promoters that may be used to
drive inducible expression of RNAi.
Inducible Promoter

Gamma Interferon-Inducible Promoters
LacSwitch inducible promoters
streptogramin-inducible promoters
Ecdysone-inducible promoters
interferon-inducible promoters
Cold-inducible promoters
Abscisic Acid-Inducible Promoters
Banana pathogen inducible promoters
Auxin-inducible promoters
ethylene-inducible promoters
radiation-inducible promoters
superoxide-inducible promoters

TABLE 2C
List of genes with promoters that can be utilized to drive
tissue and/or cell specific expression of RNAi

| No. | Gene | Associated disorder | OMIM Reference |
|---|---|---|---|
| 1 | GLC1A promoter | Glaucoma | 137750 |
| 2 | ETM1 promoter | Essential trmor | 190300 |
| 3 | Pax3 promoter | Waardenburg syndrome | 193500 |
| 4 | HD promoter | Huntington disease (HD) | 143100 |
| 5 | EVC promoter | Ellis-van Creveld syndrome | 225500 |
| 6 | Alpha-synuclein promoter | Parkinson disease | 600116 |
| 7 | NOG & BMP promoters | Fibrodysplasia Ossificans Progressiva (FOP) | 135100 |
| 8 | 5-alpha reductase promoter | Male pattern baldness | 184753 |
| 9 | CSA & CSB promoters | Cockayne syndrome | 216400 |
| 10 | SMN1 promoter | Spinal Muscular Atrophy (SMA) | 600354 |
| 11 | DTD promoter | Diastrophic dysplasia (DTD) | 222600 |
| 12 | SCA1 promoter | Spinocerebellar ataxia | 164400 |
| 13 | IDDM1 promoter | Diabetes | 222100 |
| 14 | EPM2A promoter | Epilepsy | 604223 |
| 15 | LIM kinase & elastin promoter | Williams syndrome | 194050 |
| 16 | CFTR promoter | Cystic fibrosis | 602421 |
| 17 | PDS promoter | Pendred syndrome | 274600 |
| 18 | Leptin promoter | Obesity | 164160 |
| 19 | Myc promoter | Burkitt lymphoma | 113970 |
| 20 | Frataxin promoter | Friedreich's ataxia (FRDA) | 229300 |
| 21 | ABC1 promoter | Tangier disease | 205400 |
| 22 | PAHX promoter | Refsum disease | 266500 |
| 23 | OAT promoter | Gyrate atrophy | 258870 |
| 24 | LQT1 promoter | Long QT syndrome (LQTS) | 192500 |
| 25 | VMD2 promoter | Best disease | 153700 |
| 26 | PXR1 promoter | Zellweger syndrome | 600414 |
| 27 | ATP7B promoter | Wilson's Disease | 248600 |
| 28 | SERPINA1 promoter | Alpha-1-antitrypsin (AAT) | 107400 |
| 29 | SNRPN promoter | Prader-Willi syndrome (PWS) | 176270 |
| 30 | UBE3A promoter | Angelman syndrome (AS) | 105830 |
| 31 | HBA1 and HBA 2 promoters | Thalassemia | 604131 |
| 32 | FMF promoter | Familial Mediterranean fever (FMF) | 249100 |
| 33 | APKD promoter | Polycystic kidney disease (APKD) | 173900 |
| 34 | CD19 promoter | Crohn's disease | 266600 |
| 35 | PMP22 promoter | Charcot-Marie-Tooth disease (CMT) | 118200 |
| 36 | p53 promoter | Cancer | 191170 |
| 37 | NP-C promoter | Niemann-Pick: | 257200 |
| 38 | DPC4 (Smad4) promoter | Pancreatic cancer | 606856 |
| 39 | IL2RG, JAK3, ADA promoters | Severe combined immunodeficiency (SCID) | 202500 |
| 40 | APOE promoter | Atherosclerosis | 107741 |
| 41 | BCKDH promoter | Maple Syrup Urine Disease (MSUD) | 248600 |
| 42 | MD promoter | Myotonic dystrophy | 160900 |
| 43 | AIRE promoter | Autoimmune polyglandular syndrome | 240300 |
| 44 | SOD1 promoter | Amyotrophic lateral sclerosis (ALS) | 105400 |
| 45 | SGLT1 promoter | Glucose Galactose Malabsorption (GGM) | 182380 |
| 46 | DGS promoter | DiGeorge syndrome | 188400 |
| 47 | NF2 promoter | Neurofibromatosis | 101000 |
| 48 | COL4A5 promoter | Alport syndrome (AS) | 301050 |
| 49 | PIG-A promoter | Paroxysmal nocturnal hemoglobinuria | 311770 |
| 50 | Dystrophin promoter | Duchenne muscular dystrophy (DMD) | 310200 |
| 51 | MNK promoter | Menkes syndrome | 309400 |
| 52 | MeCP2 promoter | Rett syndrome (RTT) | 312750 |
| 53 | ALD promoter | Adrenoleukodystrophy (ALD) | 300100 |

Gene expression of the RNAi and cleaving element can be limited partially or completely to specific tissue or cell types by using tissue or cell specific promoter(s) and/or other control sequences (e.g., post-transcriptional), such as, for example, enhancer sequences.

In one embodiment, the tissue specific regulation of the cleaving element and the RNAi may be achieved on different vectors, that is, the cleaving element may be expressed on one vector and the RNAi expressed on another vector. The invention therefore enables tissue specific and/or cell specific expression of RNAi in a cell, animal or plant.

In preferred embodiments of the invention, the RNAi is placed at or close to the TSS(s) of the promoter(s). In this way the number of nucleotides transcribed that are not part of the RNAi may be minimized thereby optimizing the structure of the expressed RNAi. The sequence of the promoters described and/or the sequence around the TSS(s) utilized by the promoters may be modified such that the number of nucleotides transcribed that are not part of the RNAi are minimized, thereby optimizing the structure of the expressed RNAi.

Further multiple cassettes driving tissue specific and/or cell specific and/or inducible expression of RNAi and using different TSS(s) utilized by the same promoter can be used in the invention.

Promoters for use in the practice of the invention may be inducible promoters. Such promoters are well known in the art and include for example, tetracycline inducible promoters, hyperthermia-inducible human heat shock protein-70 (hsp70) promoter, glial fibrillary acidic protein (GFAP) promoter and human interferon (IFN)-inducible MxAX (Table 2) (Sakai, 2002; Utomo, 1999). Such promoters enable inducible expression of RNAi constructs with one or more cis-acting cleaving elements that cleave 5′ and/or 3′ of the RNAi.

In another aspect of the invention the promoters utilized may be chimeric promoters combining various elements to achieve tissue specific and/or cell specific and/or temporal specific and/or level specific and/or inducible expression of RNAi.

Transcription Termination Sequences

In particular embodiments of the invention, regulatory sequences that exert post-transcriptional control on RNAi expression are included such as, e.g., intronic sequences and polyadenylation sequences.

In one embodiment of the invention termination signals are utilized 3′ of the sequence encoding RNAi to terminate transcription. Polymerase II termination signals such as polyadenylation signals may be used to terminate transcription. Various polyadenylation signals have been defined from a wide range of species and minimal polyadenylation signals can be used to terminate transcription.

In one embodiment, a tissue specific and/or cell specific and/or inducible promoter together with a 5′ cis-acting ribozyme and sequence encoding RNAi and a short termination signal 3′ of the encoded RNAi sequence may be used to achieve controlled expression of RNAi.

Cleaving Elements

In a preferred embodiment, the polynucleotide further comprises a cleaving element that is capable of cleaving a nucleotide sequence 5′ and/or 3′ to the RNAi sequence. Any suitable cleaving element that can result in sequence specific cleavage of the target RNA can be used in the invention. The cleaving element may be, for example, a ribozyme, a maxizyme, a minizyme or a DNAzyme. Preferably, the cleaving element is a ribozyme. The cleaving element is preferably located 5′ and/or 3′ of the sequence to be expressed. Cleaving elements can be located between the sense and the antisense arms of the RNAi. The cleaving element can itself be located either 5′ and/or 3′ of the termination signal(s) for transcription.

The cleaving element may cleave the RNAi so as to leave the RNAi ends flush and/or such that short overhang(s) of nucleotides (e.g., from 1-30 nucleotides, preferably less than 10 nucleotides, more preferably less than 5 nucleotides, most preferably 1 or 2 nucleotides) are generated.

The cleaving element is preferably operatively linked to a tissue specific-promoter, a cell-specific promoter and/or an inducible promoter. Although in some embodiments of the invention the promoter to which the cleaving element is operatively linked is a different promoter than that to which the RNAi is operatively linked, in preferred embodiments of the invention, the cleaving element is operatively linked to the same promoter.

In yet a further embodiment of the invention, a combination of at least one RNAi and at least one cis-acting cleaving element and at least one suppression agent (e.g., such as a trans-acting ribozyme) may be used to effect suppression of one or more target gene(s) or nucleotide sequences.

In another embodiment the polynucleotide further comprises one or more cleaving elements that is capable of cleaving a nucleotide sequence 5′ and/or 3′ and/or between the sense and the antisense strands of the RNAi sequence.

In one embodiment the sense and antisense strands of RNAi are expressed each using a promoter and sequences encoding sense and antisense arms of RNAi are present on the same and/or different constructs. Promoters to express sense and antisense strands of RNAi can be the same and/or different promoters. Antisense and sense expression cassettes can have 5′ and/or 3′ cleaving element(s).

A ribozyme can be designed to cleave an RNAi molecule by designing specific ribozyme arms that bind to a particular RNA on either side of a consensus NUX site, 5′ and/or 3′ to the RNAi sequence, where N is selected from the group consisting of C, U, G, A and X is selected from the group consisting of C, U or A. Thus, any RNA sequence possessing an NUX site is a potential target. However, other variables require consideration in designing a ribozyme, such as the two dimensional conformation of the RNAi containing the nucleotides that are to be cleaved by the ribozyme (e.g., loops) and the accessibility of a ribozyme for its target. The utility of an individual ribozyme designed to target an NUX site in an open loop structure of transcripts comprising the RNAi will depend in part on the robustness of the RNA open loop structure. Robustness may be evaluated using an RNA-folding computer program such as RNAPlotFold. A robust loop refers to the occurrence of the loop for most or all of the plotfolds with different energy levels. Robustness of loop structures is evaluated over a broad energy profile, depending on the length of the sequence, according to art known parameters.

While various agents such as ribozymes that cleave RNA at site specific recognition sequences can be used to cleave RNAi, the use of hammerhead ribozymes is preferred. Hammerhead ribozymes are small catalytic RNA enzymes that can elicit sequence specific cleavage of a target RNA transcript. Hammerhead ribozymes cleave RNAs at locations dictated by flanking regions that form complementary base pairs with the target RNA. The target RNA has the following sequence of two bases: 5′-UX-3′ where X=A, C or U. The construction and production of hammerhead ribozymes is well known in the art.

Ribozymes for use in the present invention also include RNA endoribonucleases (hereinafter “Cech-type ribozymes”) such as the one that occurs naturally in Tetrahymena Thermophila (known as the IVS, or L-19 IVS RNA). The Cech-type ribozymes have an eight base pair active site that hybridizes to a target RNA sequence and cleaves of the target RNA. The invention encompasses those Cech-type ribozymes that target eight base-pair active site sequences that are present in a target allele. Antisense arms can vary in length from 1-5 bases, from 5 to 10 bases and from 10-30 bases. Hairpin, hammerhead, trans-splicing ribozymes and indeed any ribozyme could be used in the practice of the invention. In addition, any RNA inactivating or RNA cleaving element that is capable of recognition of, and/or binding to, specific nucleotide sequences in an RNAi (e.g. splicesome-mediated RNA trans-splicing) is contemplated. In an embodiment, the cleavage of RNAi by the at least one ribozyme or other cleaving element allows the RNAi to adopt an optimal structure (e.g., secondary or tertiary) subsequent to cleavage. Suppression agents of the invention also include minizymes, maxizymes, DNAzymes and/or any other suppression agent(s) able to cleave a target RNA in a sequence specific manner.

The ribozymes can be composed of modified oligonucleotides (e.g., for improved stability, targeting, etc.). Modified oligonucleotide can be transfected into cells expressing RNAi. A preferred method of delivery involves using a DNA construct “encoding” the ribozyme under the control of a strong tissue specific, cell specific or inducible promoter, so that transfected cells will produce sufficient quantities of the ribozyme to cleave the RNAi. Because ribozymes are catalytic, a intracellular concentration of ribozymes lower than that required for antisense molecules may be sufficient for efficient cleavage.

Vectors

The RNAi can be delivered as naked DNA, modified DNA, naked RNA or in a carrier vehicle or vector. Naked nucleic acids or nucleic acids in vectors can be delivered with lipids or other derivatives which aid gene delivery. Nucleotides may be modified to render them more stable, for example, resistant to cellular nucleases while still supporting RNaseH mediated degradation of RNA or with increased binding efficiencies. Cationic lipid-mediated delivery of suppression vectors, soluble biodegradable polymer-based delivery, or electroporation/ionthophoresis may also be used. Delivery may be in vivo or ex vivo to cells.

Vectors for use in the invention may be viral, non-viral, an artificial chromosome or any vehicle for delivery of the RNAi nucleotides. Exemplary viral vectors useful in the practice of the invention include those derived from adenovirus; adenoassociated virus; retroviral-C type such as MLV; lentivirus such as HIV or SIV; herpes simplex (HSV); and SV40. Exemplary, non-viral vectors useful in the practice of the invention include bacterial vectors from Shigella flexneri, such as the S. flexneri that is deficient in cell-wall synthesis and requires diaminopimelicacid (DAP) for growth. In the absence of DAP, recombinant bacteria lyse in the host cytosol and release the plasmid.

In an embodiment, vector constructs can include more than one RNAi nucleotide sequence, wherein each RNAi may target either the same or different target genes or target nucleotide sequences.

Vectors encoding a tissue specific and/or cell specific and/or inducible RNAi may be delivered alone or with one or more agent(s) to aid delivery of constructs and/or nucleotides. Nucleic acids encoding at least one RNAi and/or ribozyme for suppression of gene expression may be provided in the same vector or in separate vectors.

Cells

The invention provides host cells comprising a polynucleotide encoding an RNAi operatively linked to a tissue specific, cell specific, and/or inducible promoter. The polynucleotide may further comprise a vector.

The invention can be practiced in any cell or tissue for which there is a tissue specific or cell specific and/or inducible promoter that can drive transcription of a nucleotide sequence. For example, the 661 W and Y79 cell lines are photoreceptor-derived cell lines (Crawford, M. et al. Biochem Biophys Res Commun 281:536 (2001)). Cone photoreceptor-specific promoter sequences such as, e.g., the GNAT-2 and peripherin-rds promoters, can be used to drive expression of transfected genes or nucleotide sequences in these cell lines. Similarly, liver specific promoters such as the albumin promoter can be used to drive expression in liver-derived cells, for example, hepatocytes. Similarly, Collagen 1A1 and 1A2 promoters can drive expression in mesenchymal progenitor stem cells and osteoblasts. Tissues and cell types in which the invention can be practised are, but are not limited to, lymphocytes, haemopoietic cells, keratinocytes, fibroblasts, chondrocytes, epithelial cells, stem cells, kidney cells, pancreatic cells, lung cells, hepatocytes, astrocytes, oliogodendrocytes, muscle cells, brain cells, neuronal stem cells, retinal stem cells, bone cells, heart cells, colon cells, intestinal cells and skin cells.

Transgenic Organisms

In an embodiment, the invention provides transgenic plants or animals, e.g., non-human animals, birds, reptiles, marsupials or amphibians, in which one or more of the cells of the animal contain heterologous nucleic acid introduced by way of human intervention, such as by transgenic techniques well known in the art. For example, nucleic acid encoding an RNAi and a cleaving element may be introduced into the cell, directly or indirectly by introduction into a precursor of the cell, by way of deliberate genetic manipulation, such as by microinjection or by infection with a recombinant virus. This molecule may be integrated within a chromosome, or it may be extrachromosomally replicating DNA.

Pharmaceutical Compositions

Pharmaceutical compositions according to the present invention, and for use in accordance with the present invention may comprise, in addition to active ingredient, a pharmaceutically acceptable excipient, carrier, buffer stabiliser or other materials well known to those skilled in the art. Such material should be non-toxic and should not interfere with the efficacy of the active ingredient. The precise nature of the carrier or other material will depend on the route of administration, which may be, for example, oral or intravenous or by other routes of injection. Delivery of the polynucleotides of the invention may be by local or systemic injection, for example.

Examples of the techniques, formulations and protocols mentioned and other techniques, formulations and protocols that may be used in accordance with the invention can be found in Remington's Pharmaceutical Sciences, 16th edition, Oslo, A. (ed), 1980.

Uses

Tissue specific gene expression systems are useful inter alia as a research tool, in the design, generation, evaluation, and implementation of therapies and in the design, generation and evaluation of genetically modified (e.g., transgenic) plants and animals, for example.

The invention may be also used to direct the suppression (e.g., either partial or complete) of a target gene or nucleotide sequence (e.g., endogenous or exogenously introduced) in a cell specific or tissue specific manner and/or in a inducible manner, thereby to study gene function, e.g., the biological function of the target gene(s), target nucleotide sequence cleaving elements or RNAis in cells, animals, or plants. Tissue specific, cell specific, or inducible expression may also be used to examine the regulation of biosynthetic pathways and to identify and characterize the participants in those pathways.

In addition, the invention may be used to generate genetically modified or transgenic animals and plants that express an RNAi and/or cleaving element in certain cells or tissues, or in response to certain stimuli.

In a further embodiment of the invention, the RNAi can be constructed to target a reporter gene to study gene expression in a tissue specific, cell specific and/or inducible manner. The use of reporter genes to study gene expression is well known to a skilled artisan (Gardner, D. P. et al. (1996) Transgenic Res. 5(1): 37-48; Hadjantonakis, A. K. et al., (2001) Histochem. Cell. Biol. 115(1): 49-58). Useful reporter genes include, for example, βgalactosidase, luciferase, green fluorescent protein (gfp) or enhanced green fluorescent protein (egfp). Vectors containing RNAi nucleotide sequences that target reporter nucleotide sequences and that are flanked by at least one cis-acting cleaving element capable of cleaving 5′ and/or 3′ of the RNAi can be constructed such that both the RNAi and the cleaving element are driven by a tissue specific promoter. For example, a vector construct containing an RNAi that targets egfp driven by a cone specific GNAT-2 or peripherin-rds promoter and containing a sequence for a cis-acting ribozyme(s) capable of cleaving 5′ and/or 3′ of the RNAi can be transfected into Y79 and/or 661W cells that transiently express the target egfp reporter gene and/or can be introduced into Y79 and/or 661W cells that have been engineered to stably express the target egfp reporter gene. In parallel, the same constructs can be evaluated in cell lines such as, e.g., COS-7 cells, 3T3 cells, 293 cells, hepatocytes, osteoblasts, neuronal cells, mesenchymal progenitor cells (MPC) cells, photoreceptors, retinal pigment epithetial cells, embryonic stem (ES) cells and many other cell types, or cell lines, some of which, for example, do not express sequences driven by either the GNAT-2 or the peripherin-rds promoters, in order the ensure the tissue specific nature of the promoter. Transfection and selection and assessment of transfectants is performed according to standard protocols.

In one embodiment of the invention, reporter genes are used to test the tissue specificity of a given promoter sequence in a cell line in which the promoter is normally active to assess the expression profile of the promoter prior to generating ribozyme-RNAi constructs with the promoter. For promoter sequences which have not been well characterised or for promoter sequences that are derived from or have components from multiple sources (chimeric promoters) the TSSs may not be clearly identified. RNAi can be used to characterize the TSSs of promoters.

In another embodiment, RNAi constructs with one or more cis-acting ribozymes, the expression of which is driven by tissue specific promoter(s), may further include one or more additional copies of the RNAi targeting egfp sequence and flanked by one or more cis-acting ribozymes. In this embodiment, the expression of the additional RNAi is under the control of an inducible promoter such as the tetracycline inducible promoter (Sakai, Molecular Pharmacol 61: 1453 2002; Utomo, Nature Biotech 17:1091 1999). Constructs containing RNAi flanked by cis-acting ribozymes and driven by an inducible promoter can act as a control to demonstrate that the absence of RNAi expression is due to the presence of the tissue specific promoter and not due to other factors (e.g., the inefficient transfection of the constructs). Other controls that can be included in such transfection experiments include the use of at least one RNAi targeting egfp and flanked by one or more cis-acting ribozymes that are driven by a ubiquitous promoter. By way of illustration, suppression of the target egfp gene in both Y79 cells and COS-7 cells transfected with this construct driven by a ubiquitous promoter would suggest that the absence of suppression using the tissue specific construct was due to the tissue specific nature of the promoter. In addition, RNAi flanked by one or more cis-acting ribozymes and driven by an inducible promoter can be used alone to demonstrate that the invention provides inducible production of RNAi.

While tissue specific promoter-driven expression in photoreceptor-like cells can be demonstrated using the Y79 and/or 661W cell lines, a wide variety of tissue specific promoters capable of regulating tissue specific expression of operatively associated gene or nucleotide sequence in specific cell types can be used, depending upon the desired cell type for expression. Similarly, any number of inducible promoters (e.g., cytokine or growth factor inducible) can be used to demonstrate inducible expression of RNAi.

Tissue specific, cell specific and/or inducible expression of RNAi can be demonstrated readily in animals expressing reporter genes. Administration of the RNAi vectors may be via, e.g., tail vein injection, intra peritoneal injection, intra vascular injection, intrathecal administration, intraventricular administration, intracoronary, intraocular injection, local delivery and/or ex vivo delivery of vector constructs carrying a tissue specific and/or cell specific or inducible promoter operatively linked to an RNAi and/or flanked by one or more cis-acting cleaving element. Administration may be facilitated using compounds to aid delivery of constructs and/or using physical methods, for example, electroporation and/or iontophoresis to aid delivery. The invention could be used to develop therapies for animals and humans. Alternatively, the invention could be used as research tools in the development of animal models mostly via transgenic techniques. In addition, they could be utilized in such animals to investigate the role/functions of various genes and gene products.

Complete silencing of a disease gene or allele in some instances may be difficult to achieve using RNAi. However, small quantities of disease-causing (e.g., mutant or abnormally regulated) gene product may be tolerated in some disorders. In others, a significant reduction in the proportion of a disease-causing gene product to normal gene product may result in an amelioration of disease symptoms and/or at times it can be preferable to partially silence a target gene. Hence, the invention may be applied to any genetic disease in animals where the molecular basis of the disease has been established. In addition, the strategy is applicable to modulating infectious disorders, e.g., by using a cytokine driven promoter (e.g., a promoter driven by interleukin 1 or interleukin 6 or both) or by suppressing replication of the infectious agent.

The invention may be applied in gene therapy approaches for biologically important genetic disorders affecting certain cell types or cell subpopulations. For example, the invention may also be used to suppress the expression of one or more target genes or nucleotide sequences in the design of a therapeutic for human, animal, or plant disorders such as, for example, genetic disorders, multifactorial disorders, and infectious disorders (see U.S. application Ser. No. 09/155,708, which is incorporated by reference in its entirety).

Accordingly, the invention further provides a method of gene therapy to a patient in need of treatment, said method comprising the step of administering to the patient an effective amount of a polynucleotide according to the first aspect of the invention and/or the vector according to the second aspect of the invention. The effective amount of a polynucleotide is determined first in vitro and through animal testing, for example, or other means of extrapolating a dosage as is well known in the art.

The invention can be used in the treatment of disorders/diseases due to one or more genes that act in a dominant negative manner. Suppression of the dominant negative gene may have beneficial effect(s), for example, for disorders such as retinitis pigmentosa, epidermolysis bullosa, osteogenesis imperfecta, Ehlers-Danlos syndrome, Marfan's disease, dominant negative cancers, Alzheimer's disease, motor neuron disease, poly cystic kidney disease, disorders due to poly glutamine expansions such as Huntington's chorea and many others. The invention can be used in the treatment of disorders where suppression of one or more genes modulates disease pathology. For example, suppression of pro-apoptotic genes can be protective against neurological degenerative disorders. In many cases it would be preferable to suppress expression of the target gene in part or completely only in the target tissue—the invention enables this approach. The invention can be used in the treatment of disorders where suppression of one or more genes modulates predisposition to the disease pathology. Genetic background(s) can influence the rate and progression of many disorders. The invention can be used in the suppression of gene(s) that accelerate disease pathology and/or result in additional detrimental features being associated with the disease pathology. In addition, the invention can be used to modulate predisposition to infectious disorders where suppression in part or in whole of one or more genes can alter the nature and the rate of infection of cells by a bacterium, virus, prion and/or any other infectious agent. “Treatment” or “therapy” includes any regime that can benefit a human or non-human animal. The treatment may be in respect of an existing condition or may be prophylactic (preventative treatment), such as a vaccine. Treatment may cure, alleviate, or prevent a condition.

Assays

The invention further provides methods and assays which may be used to identify candidate modulators of gene expression. In a preferred embodiment, the invention provides a method of identifying a modulator of a target gene, the method comprising the steps: providing a host cell of the invention or a transgenic organism of the invention, (ii) administering a candidate modulator to the host cell or said transgenic organism, and (iii) determining expression of the target gene in the presence of the candidate modulator.

Such methods may include techniques known to the skilled artisan such as, e.g., fluorometric analyses, microscopy, rt-PCR, real time RT PCR, Northern blot, ELISA assays and Western blot. While egfp is used to demonstrate the invention in principle, any gene or nucleotide sequence can be targeted with an RNAi in the same manner. The tissue specific, cell specific and/or inducible expression of, and suppression of gene expression by, RNAi can thereby be assessed. For example, animals to which an RNAi construct in operative linkage with a tetracycline inducible promoter has been, or is going to be, administered can also be administered tetracycline to induce RNAi expression prior to, during, or subsequent to delivery of the RNAi construct(s). In another example, RNAi constructs that target a reporter gene and that have one or more cis-acting cleaving elements and are driven by a tissue specific promoter may be administered locally or systemically into transgenic mice expressing the reporter gene. For example, an RNAi construct that targets, e.g., egfp, driven by a hepatocyte specific promoter such as, e.g., the rat albumin promoter, may be administered locally or systemically into transgenic mice expressing gfp (Hadjantonakis, A. K. et al. (2002) BMC Biotechnol. 11 2(1): 11; Hadjantonakis, A. K. et al. (1998) Mech. Dev. 76(1-2): 79-90) and tissue specific suppression of egfp (for example, in the liver) monitored using art known methods. Transgenic animals, for example, mice can be engineered using art known methods to carry tissue-specific and/or cell-specific and/or inducible RNAi constructs. For example, an RNAi construct that targets, e.g., egfp, driven by a hepatocyte specific promoter such as, e.g., the rat albumin promoter may be injected into fertilized mouse eggs and the resulting transgenic mice bred with mice expressing egfp and tissue specific suppression using RNAi targeting EGFP obtained in liver. While RNAi targeting EGFP is used in the example, RNAi targeting any eukaryotic gene could be used in the same manner.

The efficiency of RNAi construct delivery and RNAi suppression of gene expression can also be evaluated using such techniques.

### EXEMPLIFICATION

### Example 1

### Cis-acting ribozymes Utilized to Generate Functional RNAi in Cell Culture

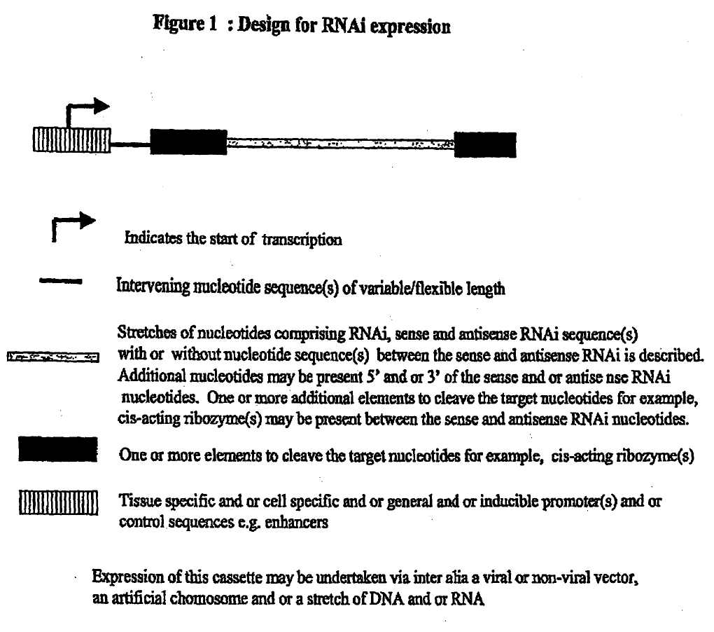

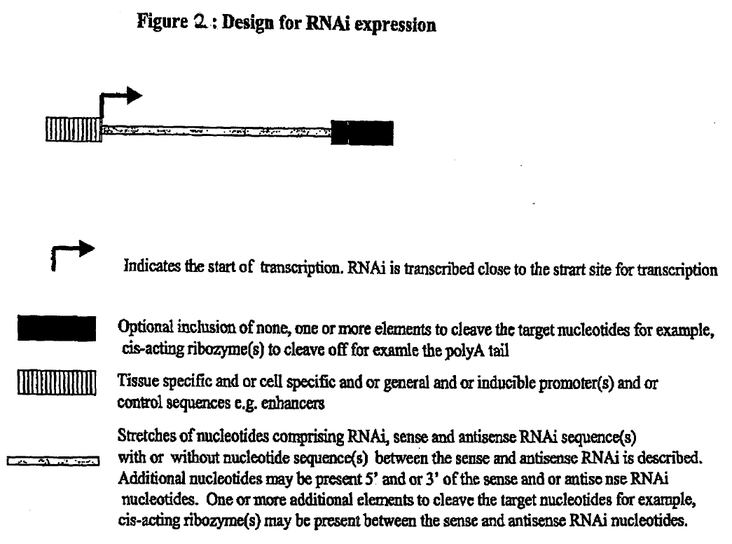

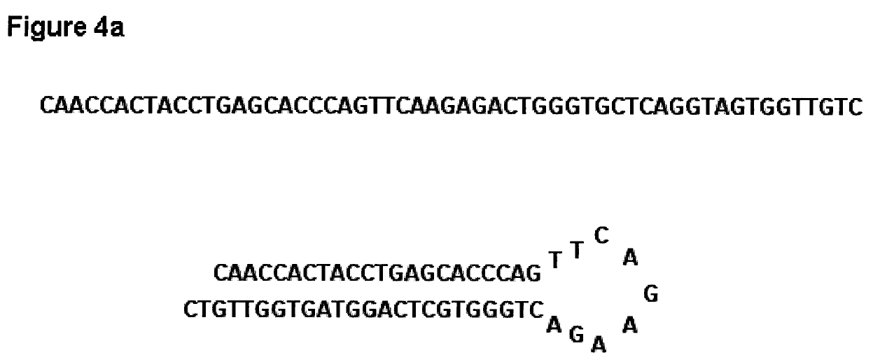

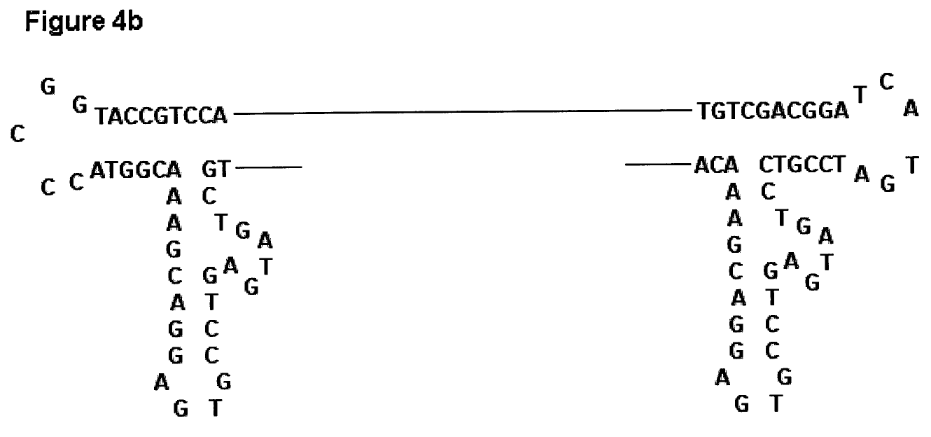

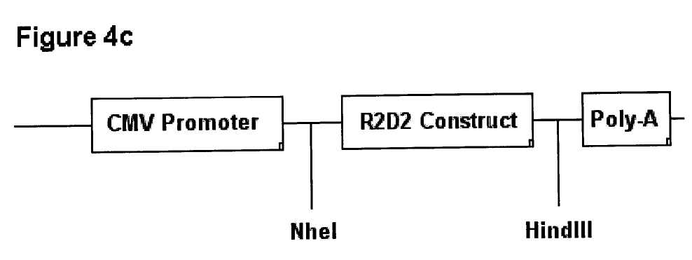

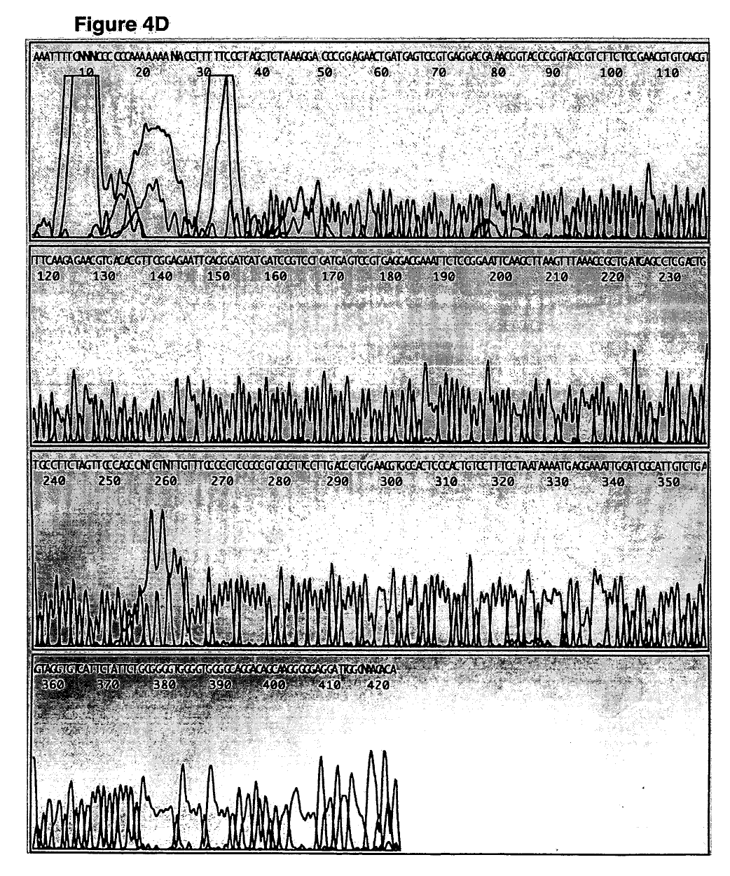

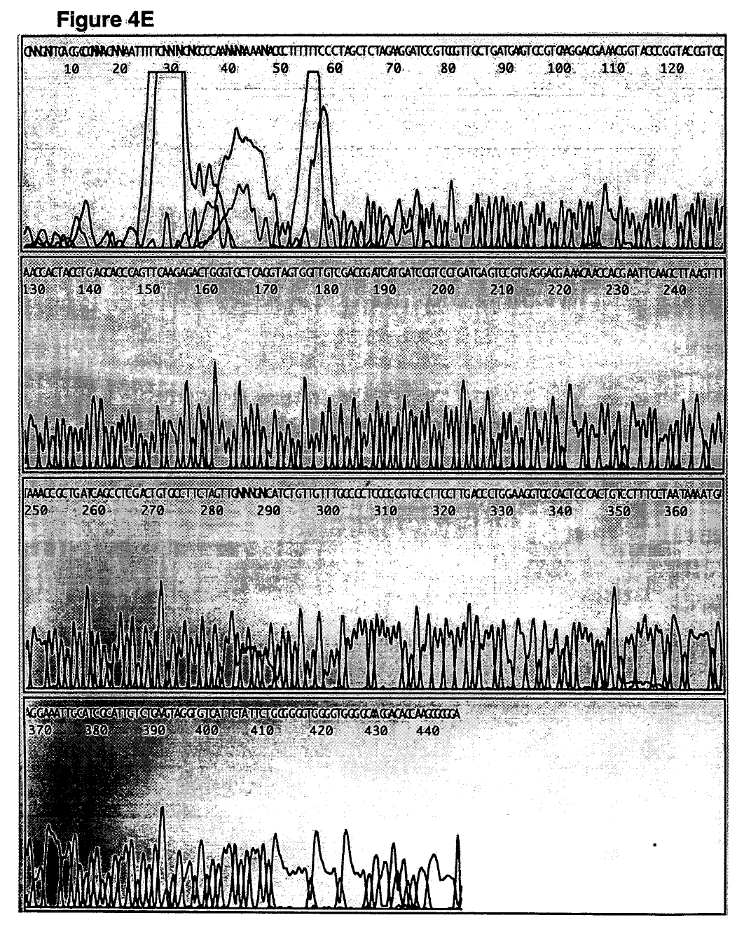

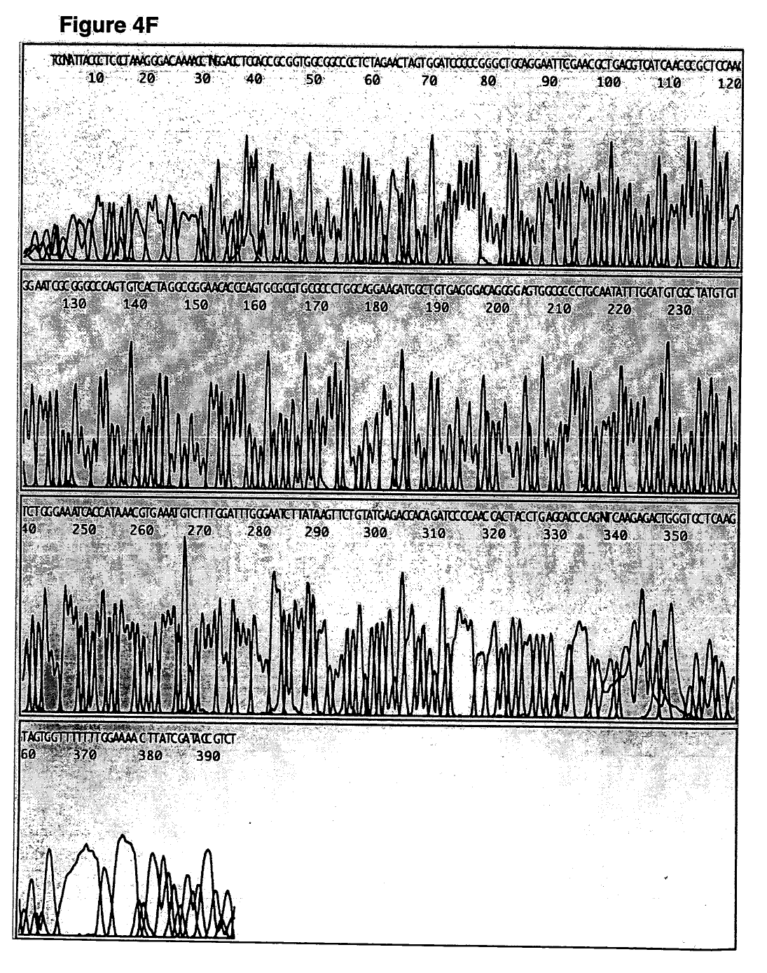

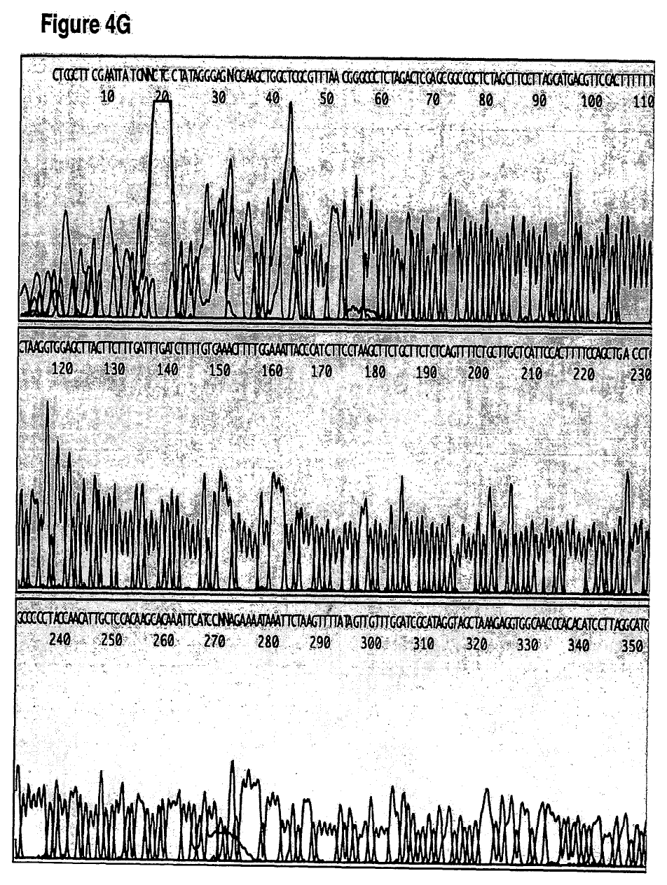

FIGS. 1A, 1B and 4 provide an overview of the method to generate RNAi expressed from a tissue specific and/or cell specific and/or inducible promoter. Table 1A and B provides sequences for RNAi targeting EGFP and a non-targeting RNAi control. The particular RNAi targeting EGFP utilized has previously been shown to be functional in cells and in vivo and has been used by Hasuwa et al. (2002) and others for this purpose. Tables 1A and 1B provides sequences for RNAi targeting EGFP flanked by 5′ and 3′ cis-acting ribozymes and a non-targeting RNAi control flanked by 5′ and 3′ cis-acting ribozymes.

TABLE 1A
RNAi sequences

R2D2egfp (SEQ ID NO: 1)
GGCTAGCTAGCTCTAGAGGATCCGTGGTTGCTGATGAGTCCGTGAGGA
CGAAACGGTACCCGGTACCGTCCAACCACTACCTGAGCACCCAGTTCA
AGAGACTGGGTGCTCAGGTAGTGGTTGTCGACGGATCATGATCCGTCC
TGATGAGTCCGTGAGGACGAAACAACCACGAATTCAAGCTTGACCTCT
CGAC

Nucleotides 6-23 are a restriction enzyme sites; nucleotides 24-30 are an arm of the ribozyme binding to hair pin RNAi; nucleotide 31-70 are a ribozyme, nucleotides 71-125 are a hairpin RNAi; nucleotides 126-166 is a ribozyme); nucleotides 167-173 is the arm of a ribozyme binding to hairpin RNAi; nucleotides 174-185 is a restriction enzyme digest site.

R2D2xera (SEQ ID NO: 2)
GGCTAGCTAGCTCTAGAGGATCCCTTGCCGCTGATGAGTCCGTGAGGA
CGAAACGGTACCCGGTACCGTCCGGCAAGCTGACCCTGAAGTTCTTCA
AGAGAGAACTTCAGGGTCAGCTTGCCGTAGACGGATCATGATCCGTCC
TGATGAGTCCGTGAGGACGAAACGGCAAGGAATTCAAGCTTGACCTCT
CGAC

Nucleotides 6-23 are a restriction enzyme sites; nucleotides 24-30 are an arm of the ribozyme binding to hair pin RNAi; nucleotide 31-70 are a ribozyme, nucleotides 71-125 are a hairpin RNAi; nucleotides 126-166 is a ribozyme); nucleotides 167-173 is the arm of a ribozyme binding to hairpin RNAi; nucleotides 174-185 is a restriction enzyme digest site.

R2D2Non (SEQ ID NO: 3)
GGCTAGCTAGCTCTAGAGGATCCCGGAGAACTGATGAGTCCGTGAGG
ACGAAACGGTACCCGGTACCGTCTTCTCCGAACGTGTCACGTTTCAAG
AGAACGTGACACGTTCGGAGAATTGACGGATCATGATCCGTCCTGATG
AGTCCGTGAGGACGAAATTCTCCGGAATTCAAGCTTGACCTCTCGAC

Nucleotides 6-23 are a restriction enzyme site; nucleotides 24-30 are an arm of the ribozyme bending to hairpin RNAi; nucleotides 31-70 are a ribozyme; nucleotides 71-119 are a hairpin RNAi; nucleotides 126-160 is a ribozyme; nucleotides 161-167 is the arm of a ribozyme bending to hairpin RNAi; nucleotides 168-179 is a restriction enzyme digest site.

Ribozyme-RNAi Construct Generation:

Three ribozyme-RNAi constructs are designed, PCR amplified and cloned into pCDNA3.1 (−). Two constructs contain RNAi sequences homologous to EGFP RNA, the third construct contains a non-targeting control RNAi sequence (which is not homologous to any known mammalian transcripts). 1. EGFP targeting construct 1 (R2D2xera). 2. EGFP targeting construct 2 (R2D2egfp). 3. Non-targeting construct (R2D2non) (Table 1A).

Primers for PCR amplification of RNAi sequences contain restriction enzyme sites to enable cloning of resulting DNA fragments into multiple vectors. The forward PCR primer (R2D2For-) contains Nhe1, Xba1 and BamH1 restriction enzyme sites. The reverse PCR primer (R2D2Rev) contains Hind111 and EcoR1 restriction enzyme sites. The same primers were used to PCR amplify each of the three ribozyme-RNAi constructs. Overlapping oligonucleotides were used as PCR templates for the three R2D2 ribozyme-RNAi constructs (Table 1B).

TABLE 1B

Sequence of oligonucleotides for PCR reactions and RNAi constructs are
provided:

| | |
|---|---|
| PCR amplification primers: | |
| R2D2For: GGC TAG CTA GCT CTA GAG GAT | (SEQ ID NO: 4) |
| R2D2Rev: GTC GAG AGG TCA AGC TTG AAT | (SEQ ID NO: 5) |
| EGFP targeting construct 1 (R2D2xera) | |
| R2D2xer1: GGC TAG CTA GCT CTA GAG GAT CCC TTG CCG CTG ATG AGT | (SEQ ID NO: 6) |
| CCG TGA GGA CGA AAC GGT ACC CGG TAC CGT CCG GCA AGC TGA CCC | |
| TGA AGT TCT TCA AGA GAG AAC TT | |
| R2D2xer2: GTC GAG AGG TCA AGC TTG AAT TCC TTG CCG TTT CGT CCT | (SEQ ID NO: 7) |
| CAC GGA CTC ATC AGG ACG GAT CAT GAT CCG TCT ACG GCA AGC TGA | |
| CCC TGA AGT TCT CTC TTG AAG AAC TT | |
| EGFP targeting construct 2 (R2D2egfp) | |
| R2D2egfp1: GGC TAG CTA GCT CTA GAG GAT CCG TGG TTG | (SEQ ID NO: 8) |
| CTG ATG AGT CCG TGA GGA CGA AAC GGT ACC CGG TAC CGT CCA ACC | |
| ACT ACC TGA GCA CCC AGT TCA AGA GAC TGG GT | |
| R2D2egfp2: GTC GAG AGG TCA AGC TTG AAT TCG TGG TTG TTT CGT CCT | (SEQ ID NO: 9) |
| CAC GGA CTC ATC AGG ACG GAT CAT GAT CCG TCG ACA ACC ACT ACC | |
| TGA GCA CCC AGT CTC TTG AAC TGG GT | |
| Non-targeting construct (R2D2non) | |
| R2D2non1: GGC TAG CTA GCT CTA GAG GAT CCC GGA GAA CTG ATG AGT | (SEQ ID NO: 10) |
| CCG TGA GGA CGA AAC GGT ACC CGG TAC CGT CTT CTC CGA ACG TGT | |
| CAC GTT TCA AGA GAA CGT GA | |
| R2D2non2: GTC GAG AGG TCA AGC TTG AAT TCC GGA GAA TTT CGT CCT | (SEQ ID NO: 11) |
| CAC GGA CTC ATC AGG ACG GAT CAT GAT CCG TCA ATT CTC CGA ACG | |
| TGT CAC GTT CTC TTG AAA CGT GA | |

The three R2D2 constructs generated by PCR amplification were cloned into pcDNA 3.1-3′ of the CMV promoter using Nhe1 and Hind111 restriction enzyme sites (FIGS. 4A-C). In addition, the HI promoter was cloned into pCDNA3.1 (Invitrogen) together with sequences encoding RNAi targeting EGFP (without 5′ and 3′ cis-acting ribozymes). This additional construct can be used as a positive control for EGFP suppression. pcDNA3.1-R2D2 constructs containing either EGFP targeting or EGFP non-targeting RNAi sequences flanked by 5′ and 3′ cis-acting ribozymes are evaluated in COS-7 cells stably expressing the EGFP target to demonstrate successful down-regulation of EGFP by RNAi generated from a CMV driven ribozyme-EGFP RNAi construct. COS-7 cells stably expressing the EGFP target are generated using standard methods (Example 4 provides protocols). Ribozyme-RNAi constructs and the HI RNAi construct are transfected into COS-7 cells expressing EGFP using Lipofectamine2000 (Invitrogen) to aid transfections and standard methods (Example 4). Levels of EGFP expression in COS-7 cells transfected with the constructs described above are evaluated using, for example, real time RT PCR (Example 4). The sequences of resulting constructs are provided in FIGS. 4D, 4E, 4F and 4G.

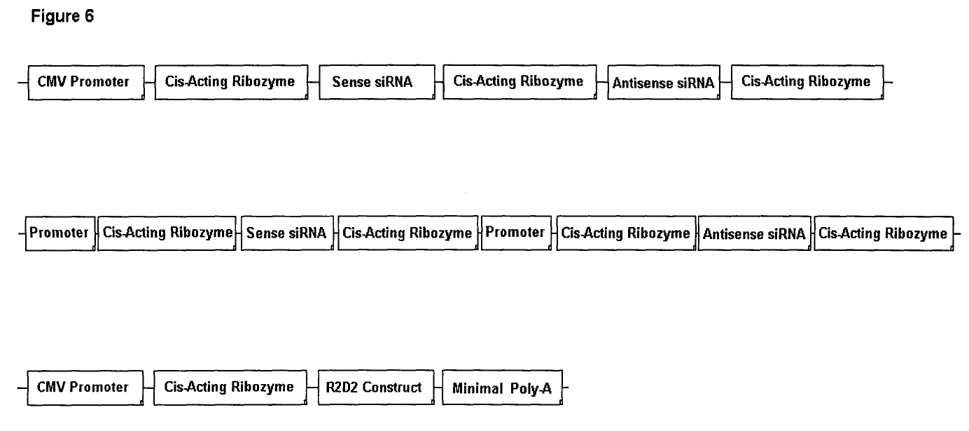

FIG. 6 shows designs for tissue specific ribozyme RNAi constructs where the sense and antisense strands of siRNA can be expressed on the same and/or different polynucleotides and can be expressed from the same and/or different tissue specific and/or cell specific and/or inducible promoters.

### Example 2

### Tissue Specific RNAi Suppression in Cell Culture

FIG. 3 provides detail on previous characterised PolII eukaryotic promoters which can drive tissue specific expression. Promoter sequences for specific genes can be used to drive tissue specific expression of a gene in one or more cell types in culture and in vivo. Many eukaryotic promoter sequences have been well defined (FIG. 3). Reporter genes such as LacZ, luciferase and GFP are used to assess the control that a given promoter sequence exerts over level and tissue specific profile(s) of gene expression. Promoters previously shown to elicit tissue specific expression of sequences placed 3′ of the promoter are cloned into vectors (for example, into pCDNA3.1 (Invitrogen) or other commercial expression vectors). Sequences encoding one or more 5′ and 3′ cis-acting ribozymes together with RNAi targeting EGFP transcripts are cloned 3′ of the promoter sequence. For some promoters the TSS(s) is not known. The use of 5′ and/or 3′ cis-acting ribozymes overcomes the need for the TSSs utilized by a tissue specific and/or cell specific and/or inducible promoter to be known.

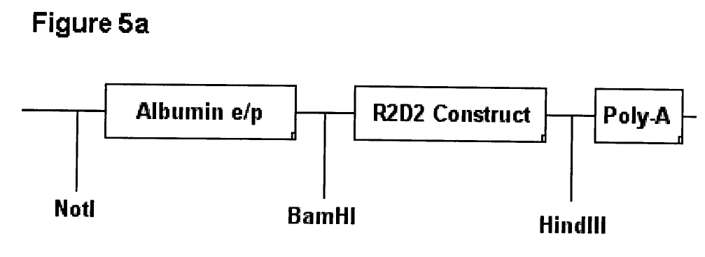

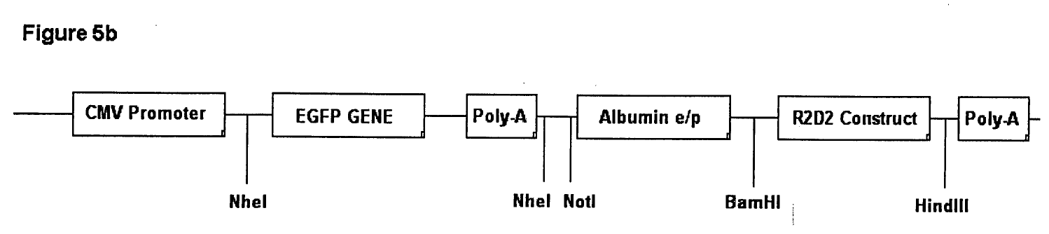

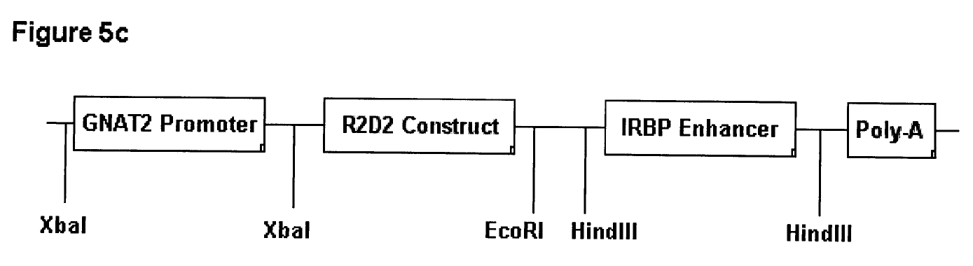

FIG. 5A shows the design of a liver specific ribozyme EGFP RNAi construct. The rat albumin promoter drives tissue specific expression in liver cells; (Postic et al. (1999) J Biol Chem 274: 305). The rat albumin promoter is active in mice expressing a promoter-gene construct. Promoters previously shown to elicit tissue specific expression of sequences placed 3′ of the promoter sequence are used to generate constructs containing RNAi targeting EGFP and non-targeting RNAi controls. Constructs also include one or more cis-acting ribozymes 5′ and/or 3′ of the RNAi sequence (see Example 1 for sequences of ribozyme-RNAi cassettes). 2.3 kb of the albumin promoter/enhancer sequence (Postic et al. J Biol Chem 274: 305 1999) is cloned into pcDNA3.1-(Invitrogen) using Not1 and BamH1 restriction enzyme sites in the multiple cloning site (MCS) of the vector. Cis-acting ribozyme-RNAi sequences (Table 1) are cloned 3′ of tissue specific promoter sequences using BamH 1 and Hind111 restriction enzyme sites (FIG. 5A).

Additional constructs are engineered (using pCDNA 3.1 or other commercial vectors) so that the target (EGFP) and the suppression agent (EGFP RNAi) are contained in the same vector. A CMV promoter and EGFP reporter gene (Clontech) and SV40 Poly-A signal are cloned (using Nhe1 restriction enzyme sites) into RNAi constructs containing a tissue specific and/or cell specific and/or inducible promoter, ribozymes and RNAi sequences. Constructs contain a CMV promoter driven EGFP gene and a tissue specific promoter driven ribozyme-RNAi cassette(s). The EGFP can be driven by any ubiquitous promoter that drives expression in most or all cell types.

The resulting constructs are used to evaluate RNAi-based EGFP down-regulation in cell culture (American Tissue Culture Collection). Hepatocyte cells lines derived from rat livers are grown in culture. Hepatocyte cells are assayed for albumin expression using rt-PCR and standard methods (Example 4). Additionally, cell lines from tissues other than liver, for example, Y79 and/or 661 W cells, photoreceptor-derived cell lines, are assayed for albumin expression by rt-PCR. Specific cell types in which the albumin gene is not expressed are confirmed. The rat albumin promoter ribozyme-EGFP-RNAi is transfected using standard procedures into hepatocyte cell lines stably expressing the EGFP target or into hepatocytes transiently transfected with a vector expressing the EGFP target (using, for example, lipofectamine and standard transfection procedures). Subsequent evaluation of RNAi-based suppression of the EGFP target is undertaken using, for example, real-time RT PCR and fluorescent microscopy (Example 4). In addition the rat albumin promoter ribozyme-EGFP-RNAi construct is transfected into cells which do not express albumin, for example, into 661 W cells, but which are stably expressing the EGFP target or transiently transfected with a vector expressing the EGFP target. Evaluation of RNAi-based suppression of the EGFP target is undertaken using inter alia real-time RT-PCR and fluorescent microscopy (Example 4).

FIG. 5C shows the design of a photoreceptor specific ribozyme EGFP RNAi construct. Photoreceptor specific expression of reporter genes can be achieved using promoters defined in cell culture and in vivo. For example, the GNAT-2 promoter and IRBP enhancer drives gene expression in cone photoreceptor cells and is expressed in a number of cone-derived cell lines (for example, Y79 cells). 280 bases of the GNAT-2 promoter and 220 bases of the IRBP enhancer (Accession Number: J03912; M22453) are cloned into the MCS of pcDNA3.1− using Xba1 restriction enzyme sites (FIG. 5) The IRBP enhancer is cloned into the same construct using BamH1 restriction enzyme sites. 5′ and 3′ cis-acting ribozyme-EGFP RNAi sequences (Table 1) are cloned 3′ of tissue specific promoter sequences using Xba1 and EcoR1 restriction enzyme sites. 661W cells and Y79 cells are assayed for GNAT-2 expression using rt-PCR and standard methods. Additionally, cell lines from tissues other than the retina, for example, from hepatocytes, liver-derived cells, are assayed for GNAT-2 expression by rt-PCR. Specific cell types in which the GNAT-2 gene is not expressed are confirmed. The GNAT-2 promoter cis-acting ribozyme EGFP RNAi constructs are transfected into photoreceptor-derived cell lines expressing GNAT-2 (for example, Y79 cells) and into non-photoreceptor-derived cell lines that do not express GNAT-2 (for example, hepatocytes). Cell lines transfected with tissue specific ribozyme RNAi constructs are engineered to stably express the EGFP target gene using standard art known methods (Example 4). In addition the EGFP gene can be transiently transfected into GNAT-2 expressing and GNAT-2 non-expressing cell lines. Subsequently levels of EGFP expression in transfected cells is evaluated using, for example, real-time RT PCR and fluorescent microscopy (Example 4). While albumin and GNAT-2 promoters are used a wide range of promoter sequences can be used, see for example, FIG. 3.

Suppression of EGFP expression is demonstrated in cell lines using tissue specific and/or cell specific and/or inducible promoters. Suppression of EGFP expression is only demonstrated when a construct containing a promoter that is active in that particular cell is utilized for transfections. Tissue specific RNAi-based suppression using eukaryotic polII promoters to drive expression of one or more 5′ and/or 3′ cis-acting ribozymes and EGFP RNAi is demonstrated.

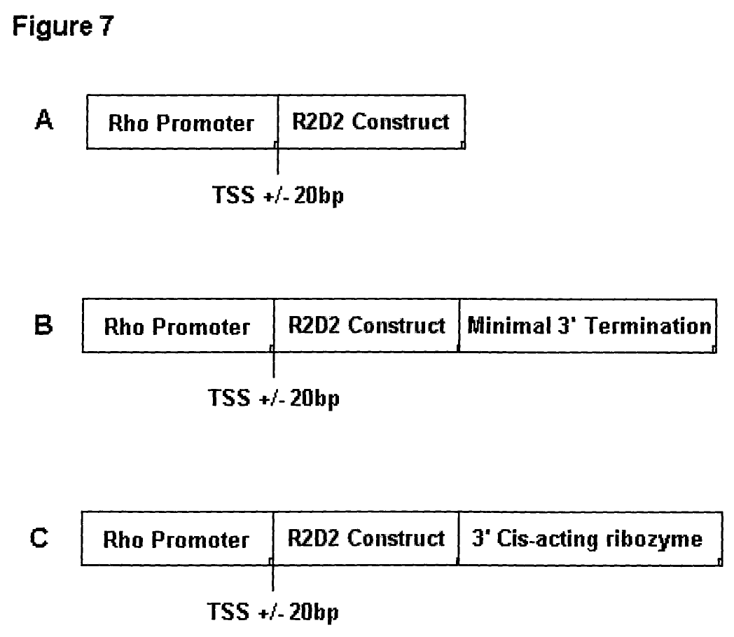

FIG. 7 shows the design of constructs where the tissue specific promoter which drives gene or nucleotide expression in photoreceptor cells is placed close to sequence encoding RNAi targeting EGFP. The RNAi is placed juxtaposed to the TSS of the rhodopsin promoter. The RNAi may also be placed close to the TSS of the tissue specific and/or inducible promoter.

### Example 3

### Inducible Promoters and Ribozyme-RNAi Constructs

5′ and/or 3′ cis-acting ribozyme-RNAi cassettes are cloned into the MCS of the pTRE2 vector (pTRE2 vector: Clontech-Cat.# 6241-1) using NheI and HindIII restriction enzyme sites. The CMV-based tetracycline responsive promoter in the vector drives expression of sequences cloned into the MCS-termination is achieved using the β-globin poly adenylation signal. 3′ of the MCS. The EGFP gene is cloned into the XhoI site 5′ of the MCS in the same vector. The Ubiquitin C promoter (370bases) is cloned into the XhoI site 5′ of the MCS in the same vector. The EGFP gene is cloned 3′ of the Ubiquitin C promoter using an artificially introduced SpeI site and the reconstructed XhoI site. The SV40 poly adenylation signal is cloned 3′ of the EGFP gene using the XhoI site.

Constructs carrying sequences encoding RNAi targeting EGFP and non-targeting control RNAi are generated. Various stable cell lines are available which constitutively express the transactivator protein required to induce expression from the tetracycline responsive promoter (Clontech). Constructs are transfected with Lipofectamine 2000 or other transfection agents into Hela cells stably expressing the transactivator protein. Transfected Hela cells are grown with and without tetracycline in the culture medium. RNA and protein is extracted from cells at various time points post transfections and levels of EGFP transcript and protein evaluated using real-time RT PCR, western blotting, ELISA and plate reader assays. The target (EGFP) and the suppression agent are in the same vector eliminating possible variability due to differing transfection efficiencies. The non-targeting control enables discrimination between possible variability in expression due to addition of tetracycline rather than the presence of functional RNAi. The inducible expression of functional RNAi released using 5′ and 3′ ribozymes is shown. The promoter used is based on the CMV promoter modified such that it is responsive to tetracycline. Other inducible promoters that respond to stimuli, for example, to chemical, electrical and/or physical stimuli can be used.

### Example 4

### Methods of Handling Cell Culture, RNA and Protein Samples and Animal Experimentation

Seeding Cells:

Cells are defrosted on ice and transferred to sterile tubes with 10 ml DMEM. Cells are pelleted at 1000 rpm (IEC Centra-3c bench top centrifuge) for 5 minutes. The supernatant is removed and the pellet resuspended in 5 ml DMEM+. A millilitre of this mix containing 0.5×10⁶ cells is placed into a 9 cm tissue culture dish and made up to 10 mls with DMEM+. Plates are incubated at 37° C. and 6% CO₂.

Splitting Cells (10 cm Dish):

Medium is removed from cells and cells washed with PBS. A millilitre of trypsin is added to the plate and the plate placed at 37° C. for 5 minutes. The plate is tapped to lift cells. DMEM+ is added to bring the volume to 10 ml. An aliquot of 2 ml is added to each new plate and again made up to 10 ml with DMEM+. Plates are incubated at 37° C. and 6% CO₂

Counting Cells (10 cm Dish):

DMEM+ is removed and the cells washed with 10 mls PBS. Two millilitres of trypsin is added and the plate is placed at 37° C. for 5 minutes. The plate is tapped to lift cells. DMEM+ is added to bring the volume to 10 ml. The mix is placed in a sterile tube and spun at 1000 rpm (IEC Centra-3c bench top centrifuge) for 5 minutes. The supernatant is removed and pellet resuspended in 1 ml DMEM+. Equal volumes of cell suspension and trypan blue are mixed (usually 10 μl of each) and placed on a haemocytometer. Sixteen squares are counted and the quantity of cells per millilitre calculated.

Freezing Down Cell Stocks:

Freezing ampoules are placed in a pre-cooled Mr. Frosty box. Cells are diluted so that 500 μl contains approximately 2×10⁷ cells. Equal volumes of cells and 2× freezing medium (500 μl of each) are added to an ampoule. Ampoules are frozen at −80° C. or placed in liquid nitrogen.

Y79 Cell Culture:

Y79 cells are cultured in suspension in RPMI medium (Gibco/BRL) supplemented with 5% Glutamine (Gibco/BRL), 5% Sodium Pyruvate (Gibco/BRL) and 10% Bovine Fetal Calf serum (Gibco/BRL). Cells are grown at 37° C. in the presence of 5% CO₂.

661W Cell Culture:

661W cone photoreceptor cells grow readily, with a doubling time of 24 hours, in Dulbecco's Modified Eagle's Medium (DMEM) with 10% (v/v) (FCS) and 2 mM L-Glutamine. Cultures are maintained in a sterile humidified environment at 37° C., 95% O₂ and 5% CO₂.

Transfection with LipofectAMINE PLUS:

Cells are counted and seeded at a density to give 50-90% confluency on the day of transfection. Volumes of DNA, reagents and media vary depending on the plate format to be used. On the day of transfection the DNA is diluted in serum free DMEM. LipofectAMINE PLUS reagent is added, mixed and incubated at room temperature for 15 minutes. Meanwhile the LipofectAMINE reagent is diluted in serum free DMEM and after 15 minutes incubation added to the DNA/LipofectAMINE PLUS mixture. This is then mixed and left at room temperature for a further 15 minutes. The media is then taken off the cells and replaced by serum free DMEM and the DNA/LipofectAMINE PLUS/LipofectAMINE mixture. Plates are incubated at 37° C. and 6% CO₂ for 3 to 5 hours. DMEM+with 30% FCS is added to bring the concentration of FCS on the cells to 10% FCS.

Transfection with Lipofectamine 2000 (Gibco/BRL):

Cells are counted and seeded at a density to give 90-95% confluency on the day of transfection. The volumes of DNA, (and RNAi), reagents and media vary depending on the plate format used. On the day of transfection the medium in the plates is replaced with antibiotic free DMEM+. The DNA is diluted in Opti-MEM I reduced serum medium. Lipofectamine 2000 reagent is diluted in Opti-MEM I reduced serum medium and after mixing is incubated for 5 minutes at room temperature. After this time the diluted DNA is added to the diluted Lipofectamine 2000 and left for a further 20 minutes at room temperature. Opti-MEM is used to bring the mixture up to its final volume. DNA/Lipofectamine 2000 complexes are added to the medium and cells. Plates are then mixed by gentle rocking and incubated at 37° C. and 6% CO₂ for 24 hours.

Transfection with Oligofectamine:

Cells are counted and seeded at a volume to give 30-50% confluence on the day of transfection. The volumes of RNAi, reagents and media vary depending on the plate format being used. On the day of transfection the medium in the plates is changed for antibiotic free DMEM+. The RNAi is diluted in Opti-MEM I reduced serum medium. Oligofectamine reagent is diluted in Opti-MEM I reduced serum medium and after mixing is incubated for 10 minutes at room temperature. After this time diluted RNAi is added to diluted Oligofectamine and left for a further 25 minutes at room temperature. Opti-MEM is used to bring the mixture up to its final volume. RNAi/Oligofectamine complexes are added to the medium and cells. Plates are then mixed by gentle rocking and left at 37° C. and 6% CO₂ for 24 hours.

Generation of Stable Cells:

Stable COS-7 cell lines expressing the EGFP target are generated using the pIRES-2 EGFP vector from Clontech-Cat.# 6029-1. Transfections for generation of stable cell lines are carried out using standard techniques with either LipofectAMINE PLUS or Lipofectamine 2000. Two days after transfection G418 selection is initiated. Media is changed every 24 hours for 3 days. G418 selection is continued for at least 4 weeks after which cells are grown without G418 or with reduced levels of G418.

Fluorescence Microscopy:

Fluorescence microscopy is undertaken using a Zeiss Axioplan 2 with a UV light source and filters. Images are analyzed by computer using the KS300 imaging system from Zeiss.

RNA Isolation from Cells:

RNAs are isolated using Trizol (Gibco/BRL) and standard procedures.

Real Time RT PCR Analysis:

Real time RT PCR is performed using the Quantitect Sybr Green RT-PCR kit. (Qiagen GmBH, Hilden). GAPDH or β-actin is used as an internal control. All primers for real time RT PCR are HPLC purified. The ROCHE lightcycler real time RT PCR machine is used in all analyses. Real time RT PCR reactions involved a denaturing step at 95° C., annealing at 55° C. and extension step at 72° C. for 34 cycles. PCR products are analyzed by electrophoresis on a 2% agarose gel.

Western Blotting:

The protein is extracted from COS-7 cells or other cells using RIPA buffer. The protein extract is quantified using the Bradford method. Equal amounts of protein are loaded and separated via SDS-PAGE. Once separation is complete the proteins are transferred to a nitrocellulose membrane using the tank blotting procedure.

The eGFP protein is detected using a Goat anti-GFP-HRP conjugate antibody (abcam663). Once the antibody has been bound and non-specifically bound antibody is washed off, chemiluminiscent detection is carried out, the blot exposed to x-ray film and an autoradiograph taken. The GFP specific band should be 42 kDa.

ELISA:

Enzyme-Linked Immunosorbent Assay (ELISA) is a useful and powerful method in estimating ng/ml to pg/ml ordered materials in solution, such as serum, urine and culture supernatant. The ELISA is based on the principle of antibody-antigen interaction. The following protocol using the GFP antibody (ab6673) from abcam facilitates quantification of EGFP.

Dilute the antigen to a final concentration of 20 μg/ml in PBS. Coat the wells of a PVC microtiter plate with the antigen by pipeting 50 μl of the antigen dilution per well. Cover the plate with an adhesive plastic and incubate for 2 h at room temperature. Remove the coating solution and wash the plate twice by filling the wells with 300 μl PBS. The solutions or washes are removed by flicking the plate over a sink. The remaining drops are removed by patting the plate on a paper towel. Block the remaining protein-binding sites in the coated wells by adding 300 μl blocking buffer, 5% non fat dry milk/PBS, per well. Cover the plate with an adhesive plastic and incubate for at least 2 h at room temperature or overnight at 4° C. Wash the plate twice with PBS. Make 10-fold dilutions (1:100, 1:1,000, 1:10,000, 1:100,000 and 1:1,000,000) of samples in blocking buffer. Add 50 μl of each dilution to an antigen-coated well. Cover the plate with an adhesive plastic and incubate for 2 h at room temperature. Wash the plate four times with PBS. Add 50 μl of secondary antispecies antibody conjugated to alkaline phosphatase, diluted at the optimal concentration (according to the manufacturer) in blocking buffer immediately before use. Cover the plate with an adhesive plastic and incubate for 2 h at room temperature. Wash the plate four times with PBS. Dissolve p-Nitrophenyl phosphate at a concentration of 1 mg/ml in substrate buffer (1M diethanolamine, 0.5 mM MgCl2, pH 9.8). Add 50 μl of the substrate solution per well with a multichannel pipet or a multipipet. Measure the absorbance at 405 nm, using a microtiter plate spectrophotometer. Perform an end-point measurement after 1 h. Calculate the titer of the sample. The titer can be defined as the dilution of serum giving an optical density (OD) of 0.2 above the background of the ELISA after a 1-h reaction.

Plate Reader Assay:

E-GFP expressing cells (verified using fluorescent microscopy) are plated out in 24 well plates (Falcon). The cells are washed in PBS and overlaid with 500 μl of PBS. The cells are excited at 485 nm and fluorescence detected at 530 nm (Wallac VICTOR multilabel plate reader).

Mouse Eye Subretinal Injection:

The mouse is anaesthetized by means of Ketamine (2.08 mg per 15 gram body weight) and Xylazine (0.21 mg per 15 gram body weight) injected intraperitoneally. The eye is proptosed and maintained in position by means of a loosely tied 10.0 nylon suture placed at the junction of the nasal 1/3^{rd} and temporal 2/3^{rd} of the upper and lower eyelids. Using a Leica Wild™ operating microscope the conjunctiva is reflected back to expose the sclera temporally. A puncture wound is made in the sclera approximately 1 mm behind the corneo-scleral limbus by means of a beveled 30-gauge needle. 3 μl of the solution to be injected is delivered subretinally by means of a 10 μl Hamilton syringe and a 30-gauge beveled needle to raise a subretinal bleb. The bleb can be visualized using the operating microscope after a drop of Vidisic™ and a small glass cover slip are placed over the cornea. The suture is removed and the eye gently replaced. The mouse is placed on a 37° C. heating pad until it recovers from the anaesthetic, after which it is replaced in the cage.

Retinal RNA Extraction:

Mouse retinas are vortexed in a solution of 500 μl Guanidinium Thiocyanate and 7.1 μl/ml β-mercaptoethanol and left overnight at room temperature. 50 μl of 2M Sodium Acetate (pH4.0), 500 μl DEPC-treated H₂O saturated Phenol and 200 μl chloroform/Isoamyl alcohol (49:1) are added to the lysate and mixed gently by inversion. The solution is left on ice for 30 minutes and centrifuged at 13,200 rpm for 20 minutes. The supernatant is transferred to a new eppendorf. 1 μl Glycogen and 1 ml of cold isopropanol is added and mixed by inversion before being left at −20° C. for 2 hours. The supernatant from a 30 minute spin is discarded and the pellet washed in 500111 of 75% ethanol. Pellets are dried at 80° C. for 3 minutes. RNA is re-suspended in 30 μl depc-treated H₂O and stored immediately at −70° C. The quality of the RNA is assessed by spectrophotometric reading of OD₂₆₀/OD₂₈₀ and also by examining 28S, 18S, and 5S bands on a 2% agarose gel.

Mouse Tail Vein Injection:

The mouse is placed inside a tube (a 50 ml syringe from which the plunger has been removed is ideal). One end is stopped by a rubber bung, in which a central hole has been cut out to act as an air channel. The other end is stopped by a rubber bung, in which a channel has been cut to allow the animal's tail to protrude. The animal's tail is gently warmed by exposure to an incandescent light bulb or by being wrapped in a cloth soaked in water at 50° C. This causes the tail veins to dilate. The veins can be readily visualized by transilluminating the tail from behind. A binocular loupe is used to magnify the view. The solution to be injected is drawn up into a 1 ml syringe to which a 30-gauge needle is attached. Taking a firm hold of the animal's tail and turning the bevel up the operator gently introduces the tip of the needle under the skin into the vein. If the plunger is gently retracted the operator can be assured that the needle is in the vein by seeing a small amount of blood enter the syringe. Using constant pressure the solution is injected at a rate of approximately 500 μl in 5 seconds. The operator should feel no resistance as the injection proceeds. If resistance is felt it indicates that extravasation into the tissues surrounding the vein has occurred. Should this happen the operator should stop the injection immediately. The remainder of the volume to be delivered can be injected at a site further up the vein. To allow for this eventuality the operator should make the first injection site as close to the end of the tail as possible. This ensures that the more proximal portion of the tail-vein remains available for injection should the first attempt fail. Following successful injection the animal is removed and returned to the cage. Any bleeding that occurs at the injection site usually stops after a very short period of time. Mice tolerate tail-vein injection well. Typically no anaesthesia is required.

Modifications on protocols for tail vein injections can be made as, for example, in Lewis et al. Nature Genetics 32: 107-108.

Iontophoresis:

Compounds are suspended in balanced saline solution (BSS) and are ionthophoresed into the eye using the transscleral CCI applicator and the method as described in (Voigt et al. Invest Ophthalmol Vis Sci 43: 3299 2002). The battery operated, microprocessor programmable CCI instrument produces a constant current (in milliamps) and uniform electrical field (in volts per square centimeter) for the treatment duration. A custom-made conical transscleral probe for rabbit (MED 6033; Nusil, Inc., CA) has an annular surface of 0.5 cm2 and an outer diameter of 17 mm, assuring its location between pars plana and limbus, with a clear opening of 13 mm to avoid contact with the cornea. Before treatment, the eye is proptosed and CCI is applied for 10 minutes at a current density of 5 mA/cm2. A peristaltic pump induces circulation under a maximum suction pressure of 25 mm Hg to ensure constant drug flow. A low-impedance, 2-cm2 custom-made rectal probe serves as the anodal return electrode, because it avoids the erratic impedance problems associated with dermal patches or subcutaneous needles in rabbits.

### Example 5

### Transgenic Annuals

Transgenic animals expressing the EGFP reporter gene from strong ubiquitous promoters such as the CMV promoter have previously been generated in the art producing so called ‘green’ animals (Jackson Laboratories). RNAi targeting EGFP has been shown to be functional in vivo in ‘green’ mice administered with commercially synthesized RNAi and/or RNAi generated from transgenes. Systemic administration of synthesized RNAi resulted in significant suppression of EGFP expression in multiple tissues of EGFP-expressing mice.

Promoter driven cis-acting ribozyme-RNAi constructs are evaluated in mice expressing the EGFP target gene. Routes of administration of tissue specific ribozyme-RNAi constructs include systemic administration, for example, by tail vein injection and/or local administration, for example, sub-retinal injection of the photoreceptor specific (GNAT-2) ribozyme-RNAi construct (Examples 2 and 4). Constructs can be administered with compounds to aid transfection efficiency and/or in viral and/or non-viral vectors. Tissues are harvested post-administration of constructs—samples are taken 0-400 days post administration of constructs. RNA is extracted from tissues and real time RT PCR undertaken. In addition, suppression of the EGFP target is evaluated in animals using western blotting, ELISA assays and fluorescent microscopy and a plate reader assay (to assay for levels of EGFP protein). Suppression of EGFP expression is demonstrated in different tissues correlating with the tissue specific promoter ribozyme RNAi constructs administered to EGFP mice.

Tissue specific promoter ribozyme RNAi constructs can be used to generate transgenic mice carrying the construct using art known methods for transgenic animal generation. Transgenic animals, for example, mice carrying the tissue specific promoter ribozyme RNAi constructs can be bred with mice expressing EGFP and suppression of EGFP expression demonstrated in specific tissues. RNA and protein samples are extracted from both tissues in which the tissue specific promoter ribozyme RNAi constructs are predicted to be expressed and from tissues in which the tissue specific promoter ribozyme RNAi constructs are predicted not to be expressed. Techniques inter alia real time RT PCR, western blotting, ELISA and the plater reader assay are used to assess EGFP suppression in various tissue samples.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

We claim:

1: A polynucleotide comprising:
a nucleotide sequence encoding an RNAi operatively linked to a tissue specific promoter, a cell specific promoter, and/or an inducible promoter.

2-17. (canceled)

18: A vector comprising a polynucleotide according to claim 1.

19 (canceled)

20: A host cell comprising the polynucleotide according to claim 1.

21-22. (canceled)

23: A transgenic animal comprising the polynucleotide according to claim 1.

24: A method of inhibiting or reducing expression of a target gene in a cell of an organism, said method comprising the steps:
(i) administering to said cell a polynucleotide according to claim 1, wherein the RNAi has specificity or partial specificity for the target gene; and
(ii) allowing expression of the RNAi such that the RNAi inhibits or reduces expression of the target gene.

25. (canceled)

26: A method of identifying a modulator of a target gene, said method comprising the steps:
(i) providing a host cell according to claim 20;
(ii) administering a candidate modulator to said host cell or said transgenic organism; and
(iii) determining expression of said target gene in the presence of the candidate modulator.

27. (canceled)

28: A pharmaceutical composition comprising a polynucleotide according to claim 1 and a pharmaceutical excipient.

29-30. (canceled)

31: A method for establishing the biological function(s) of a target gene(s) in the host cell claim 20.
